SEQUENCE LISTING (21 sequences)

SEQ ID NO: 1 - protein, 391 aa, Francisella novicida
  MUTAGEN (131)..(131): Xaa indicates Arg, Lys, His, Asn, Gln or Tyr.
  MUTAGEN (211)..(211): Xaa indicates His, Asn or Gln.
  MUTAGEN (318)..(318): Xaa indicates Ala, Cys, Gly, Ile, Leu, Met, Pro, Thr, Val, Asn, Asp, Gln or Glu.
MTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSXRVKIKSIDDVKQVLDKDSNFIIGKITLPFKKEWQRLYREWQNTTIKDDYEFLKSFFNVKSITKLHKKVRKDFSLPISTNXGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSXPKVRVKLDYVIDDDSKINYFMNHSLLKSRYPDKVLEILKQSTIIEFESSGFNKTIKEMLGMKLAGIYNETSNN

SEQ ID NO: 2 - protein, 1629 aa, Francisella novicida
  MUTAGEN (1369)..(1369): Xaa indicates Arg, Lys, His, Asn, Gln or Tyr.
  MUTAGEN (1449)..(1449): Xaa indicates His, Asn or Gln.
  MUTAGEN (1556)..(1556): Xaa indicates Ala, Cys, Gly, Ile, Leu, Met, Pro, Thr, Val, Asn, Asp, Gln or Glu.
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSXRVKIKSIDDVKQVLDKDSNFIIGKITLPFKKEWQRLYREWQNTTIKDDYEFLKSFFNVKSITKLHKKVRKDFSLPISTNXGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSXPKVRVKLDYVIDDDSKINYFMNHSLLKSRYPDKVLEILKQSTIIEFESSGFNKTIKEMLGMKLAGIYNETSNN

SEQ ID NO: 3 - DNA, 1176 bp, Francisella novicida
  misc_difference (391)..(393): nnn indicates cgt, cgc, cga, cgg, aga, agg, aaa, aag, cat, cac, aat, aac, caa, cag, tat or tac.
  misc_feature (391)..(393): n is a, c, g, or t
  misc_difference (631)..(633): nnn indicates cat, cac, aat, aac, caa or cag.
  misc_feature (631)..(633): n is a, c, g, or t
  misc_difference (952)..(954): nnn indicates gct, gcc, gca, gcg, tgt, tgc, ggt, ggc, gga, ggg, att, atc, ata, tta, ttg, ctt, ctc, cta, ctg, atg, cct, ccc, cca, ccg, act, acc, aca, ?acg, gtt, gtc, gta, gtg, aat, aac, gat, gac, caa, cag, gaa or gag.
  misc_feature (952)..(954): n is a, c, g, or t
atgactagggatggaatctatgcagagaattacctgcctatcctgattcataaggagctgaacgaagtgaggaaggggtacacatggaaaaattccgaggaaatcaaaattttcaagggaaagaaatacgacatccagcagctgaataacctggtgtattgtctgaagtttgtggacaaaccaatcagtattgatatccagatttcaaccctggaggaactgagaaacatcctgactaccaataacattgcagccactgccgagtactattacattaatctgaaaacccagaagctgcacgagtattacatcgaaaattacaacacagccctggggtataagaaatacagcaaggagatggagttcctgaggtccctggcttataggtctnnncgcgtgaagatcaaaagtattgacgatgtcaagcaggtcctggacaaggattcaaacttcatcatcggaaagatcacactgcccttcaagaaagagtggcagcgactgtaccgggaatggcagaacacaactatcaaagacgattatgagtttctgaagagcttctttaatgtgaagtccattactaaactgcacaagaaagtccggaaagacttctctctgcccatcagtacaaacnnnggcaagtttctggtgaagagaaaaacttgggataataacttcatctaccagattctgaatgactcagatagcagggcagacgggactaaacccttcattcctgcctttgatatcagcaagaacgagattgtggaagccatcattgacagtttcacctcaaaaaacatcttttggctgccaaagaatattgagctgcagaaggtggacaacaagaacatcttcgccattgataccagcaagtggtttgaggtcgaaacaccatccgacctgcgcgatatcggcattgctaccattcagtacaagatcgacaataactcannncccaaggtgcgagtcaaactggattacgtgatcgacgatgacagcaagattaactatttcatgaatcactcactgctgaagagccggtatcccgacaaagtcctggagatcctgaagcagagcacaatcattgagttcgaaagttcagggtttaacaaaactattaaggagatgctgggaatgaagctggccggcatctacaatgaaacctccaataactaa

SEQ ID NO: 4 - DNA, 4890 bp, Francisella novicida
  misc_difference (4105)..(4107): nnn indicates cgt, cgc, cga, cgg, aga, agg, aaa, aag, cat, cac, aat, aac, caa, cag, tat or tac.
  misc_feature (4105)..(4107): n is a, c, g, or t
  misc_difference (4345)..(4347): nnn indicates cat, cac, aat, aac, caa or cag.
  misc_feature (4345)..(4347): n is a, c, g, or t
  misc_difference (4666)..(4668): nnn indicates gct, gcc, gca, gcg, tgt, tgc, ggt, ggc, gga, ggg, att, atc, ata, tta, ttg, ctt, ctc, cta, ctg, atg, cct, ccc, cca, ccg, act, acc, aca, ?acg, gtt, gtc, gta, gtg, aat, aac, gat, gac, caa, cag, gaa or gag.
  misc_feature (4666)..(4668): n is a, c, g, or t
atgaattttaagatcctgcctatcgcaatcgacctgggcgtcaagaatactggcgtgtttagtgctttttatcagaaggggacctcactggagagactggacaataagaacggaaaagtgtatgaactgtccaaggattcttacactctgctgatgaacaataggaccgcacggagacaccagaggcgaggaattgacaggaaacagctggtgaagcgcctgttcaaactgatctggacagagcagctgaacctggaatgggataaggacactcagcaggccatcagcttcctgtttaatcgacggggattctcttttattactgacggctatagtcctgagtacctgaacatcgtgccagaacaggtcaaggcaatcctgatggacattttcgacgattataatggcgaggacgatctggattcctacctgaaactggccacagagcaagagagtaagatcagcgaaatctacaacaagctgatgcagaagatcctggagttcaagctgatgaaactgtgcaccgacatcaaggacgataaagtgagtaccaagacactgaaagagatcacaagctacgagttcgaactgctggccgattatctggctaactacagcgaatccctgaagacccagaaattttcctacacagacaagcagggcaatctgaaagagctgtcttactaccaccatgataagtacaacatccaggagttcctgaagagacacgccaccatcaatgacaggattctggatacactgctgactgacgatctggacatctggaacttcaacttcgagaagttcgatttcgacaagaacgaggaaaaactgcagaatcaggaagataaggaccacattcaggctcatctgcaccatttcgtgtttgcagtcaataagatcaaaagcgagatggcatccggcgggcgccatcgaagccagtacttccaggaaatcaccaacgtgctggacgagaacaatcaccaggaaggctacctgaaaaacttctgtgagaatctgcataacaagaagtacagcaatctgtccgtgaagaatctggtcaacctgattggaaatctgtccaacctggaactgaagcccctgcgcaaatacttcaacgacaagatccacgctaaagcagaccattgggatgagcagaagtttactgaaacctattgccactggattctgggcgagtggcgggtgggggtcaaggatcaggacaagaaagacggcgcaaagtattcttacaaggacctgtgtaacgagctgaagcagaaagtgactaaggccgggctggtggacttcctgctggagctggacccctgccgaaccattccaccttacctggacaacaataacagaaagccacccaaatgtcagagcctgatcctgaatcccaagtttctggataatcagtatcctaactggcagcagtacctgcaggagctgaagaaactgcagtcaatccagaactacctggacagcttcgaaaccgatctgaaggtgctgaaaagctccaaggaccagccttacttcgtcgagtacaagtctagtaaccagcagatcgcttccggccagcgggattacaaggatctggacgcaagaatcctgcagttcatttttgacagggtgaaggcctctgatgagctgctgctgaacgaaatctatttccaggcaaagaaactgaagcagaaagcctcaagcgagctggaaaagctggagtcctctaagaaactggacgaagtgatcgctaactctcagctgagtcagattctgaagtctcagcacacaaatggaatcttcgagcagggcacttttctgcatctggtgtgcaaatactataagcagcgacagagagccagggacagccgcctgtacatcatgcctgaatatcgatacgataagaaactgcacaagtacaacaacaccggccgctttgacgatgacaaccagctgctgacatattgtaatcataagccccggcagaaaagataccagctgctgaacgacctggcaggagtgctgcaggtctctcctaattttctgaaggataaaatcgggtccgatgacgatctgttcatttctaagtggctggtggagcacatccggggctttaagaaggcctgcgaagacagcctgaaaatccagaaggataacaggggactgctgaatcataagatcaacattgcacgcaataccaagggcaaatgcgagaaagaaatcttcaacctgatctgtaagattgaggggagcgaagacaagaaagggaattataagcacggactggcctacgagctgggagtgctgctgttcggagagccaaacgaggccagcaagcccgaatttgataggaaaatcaagaaattcaattcaatctacagctttgcccagatccagcagattgcctttgctgagaggaaggggaatgcaaacacatgcgccgtgtgtagtgcagacaacgcccatcgcatgcagcagatcaaaattactgagccagtcgaagacaataaggataaaatcattctgtcagcaaaggcacagcgactgcctgcaatcccaacccgaattgtggatggagctgtcaagaaaatggctacaattctggcaaagaatatcgtggacgataattggcagaacattaagcaggtcctgagcgcaaaacaccagctgcatatcccaatcattaccgagtccaacgccttcgagtttgaacccgctctggcagacgtgaagggcaaatctctgaaggatagaaggaagaaagccctggagcgaattagtcccgaaaacatcttcaaggataagaacaacagaatcaaggagtttgctaaggggatttccgcctactctggagctaacctgacagatggggacttcgatggagcaaaggaggaactggatcacatcattcctcgcagccataagaaatatggcactctgaacgacgaggctaatctgatttgcgtgacccggggcgataataagaacaaagggaaccggatcttctgtctgagagacctggccgataattacaagctgaaacagtttgagaccacagacgatctggagatcgaaaagaaaattgccgacaccatctgggatgctaataagaaggacttcaagttcggaaactatcggagcttcatcaatctgacacctcaggagcagaaagcattcagacacgccctgtttctggctgatgaaaacccaatcaagcaggcagtgatcagagccattaataaccgcaaccgaaccttcgtgaatggcacacagaggtattttgctgaggtcctggcaaataacatctacctgcgcgccaagaaagaaaatctgaacactgacaagatcagcttcgattactttggaatccctaccattggaaacggccgagggatcgctgagattcggcagctgtatgaaaaggtggacagtgatatccaggcctacgctaaaggcgacaagccacaggcctcttatagtcacctgattgatgctatgctggcattctgcatcgccgctgacgagcatcggaacgatggatctattggcctggaaatcgacaaaaactatagtctgtaccctctggataagaatactggcgaggtgttcaccaaagacatcttttcacagatcaagattaccgacaacgagttcagcgataagaaactggtcagaaagaaagctattgaagggtttaacacacacagacagatgactagggatggaatctatgcagagaattacctgcctatcctgattcataaggagctgaacgaagtgaggaaggggtacacatggaaaaattccgaggaaatcaaaattttcaagggaaagaaatacgacatccagcagctgaataacctggtgtattgtctgaagtttgtggacaaaccaatcagtattgatatccagatttcaaccctggaggaactgagaaacatcctgactaccaataacattgcagccactgccgagtactattacattaatctgaaaacccagaagctgcacgagtattacatcgaaaattacaacacagccctggggtataagaaatacagcaaggagatggagttcctgaggtccctggcttataggtctnnncgcgtgaagatcaaaagtattgacgatgtcaagcaggtcctggacaaggattcaaacttcatcatcggaaagatcacactgcccttcaagaaagagtggcagcgactgtaccgggaatggcagaacacaactatcaaagacgattatgagtttctgaagagcttctttaatgtgaagtccattactaaactgcacaagaaagtccggaaagacttctctctgcccatcagtacaaacnnnggcaagtttctggtgaagagaaaaacttgggataataacttcatctaccagattctgaatgactcagatagcagggcagacgggactaaacccttcattcctgcctttgatatcagcaagaacgagattgtggaagccatcattgacagtttcacctcaaaaaacatcttttggctgccaaagaatattgagctgcagaaggtggacaacaagaacatcttcgccattgataccagcaagtggtttgaggtcgaaacaccatccgacctgcgcgatatcggcattgctaccattcagtacaagatcgacaataactcannncccaaggtgcgagtcaaactggattacgtgatcgacgatgacagcaagattaactatttcatgaatcactcactgctgaagagccggtatcccgacaaagtcctggagatcctgaagcagagcacaatcattgagttcgaaagttcagggtttaacaaaactattaaggagatgctgggaatgaagctggccggcatctacaatgaaacctccaataactaa

SEQ ID NO: 5 - protein, 829 aa, Francisella novicida
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIK

SEQ ID NO: 6 - protein, 786 aa, Francisella novicida
  MUTAGEN (526)..(526): Xaa indicates Arg, Lys, His, Asn, Gln or Tyr.
  MUTAGEN (606)..(606): Xaa indicates His, Asn or Gln.
  MUTAGEN (713)..(713): Xaa indicates Ala, Cys, Gly, Ile, Leu, Met, Pro, Thr, Val, Asn, Asp, Gln or Glu.
SAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSXRVKIKSIDDVKQVLDKDSNFIIGKITLPFKKEWQRLYREWQNTTIKDDYEFLKSFFNVKSITKLHKKVRKDFSLPISTNXGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSXPKVRVKLDYVIDDDSKINYFMNHSLLKSRYPDKVLEILKQSTIIEFESSGFNKTIKEMLGMKLAGIYNETSNN

SEQ ID NO: 7 - protein, 150 aa, Artificial Sequence
  Synthesized
HTLYAPGGYDIMGYLRQIRNRPNPQVELGPVDTSCALILCDLKQKDTPIVYASEAFLYMTGYSNAEVLGRNCRFLQSPDGMVKPKSTRKYVDSNTINTMRKAIDRNAEVQVEVVNFKKNGQRFVNFLTMIPVRDETGEYRYSMGFQCETE

SEQ ID NO: 8 - protein, 151 aa, Artificial Sequence
  Synthesized
GHTLYAPGGYDIMGYLDQIGNRPNPQVELGPVDTSCALILCDLKQKDTPIVYASEAFLYMTGYSNAEVLGRNCRFLQSPDGMVKPKSTRKYVDSNTINTIRKAIDRNAEVQVEVVNFKKNGQRFVNFLTIIPVRDETGEYRYSMGFQCETE

SEQ ID NO: 9 - DNA, 4890 bp, Francisella novicida
atgaattttaagatcctgcctatcgcaatcgacctgggcgtcaagaatactggcgtgtttagtgctttttatcagaaggggacctcactggagagactggacaataagaacggaaaagtgtatgaactgtccaaggattcttacactctgctgatgaacaataggaccgcacggagacaccagaggcgaggaattgacaggaaacagctggtgaagcgcctgttcaaactgatctggacagagcagctgaacctggaatgggataaggacactcagcaggccatcagcttcctgtttaatcgacggggattctcttttattactgacggctatagtcctgagtacctgaacatcgtgccagaacaggtcaaggcaatcctgatggacattttcgacgattataatggcgaggacgatctggattcctacctgaaactggccacagagcaagagagtaagatcagcgaaatctacaacaagctgatgcagaagatcctggagttcaagctgatgaaactgtgcaccgacatcaaggacgataaagtgagtaccaagacactgaaagagatcacaagctacgagttcgaactgctggccgattatctggctaactacagcgaatccctgaagacccagaaattttcctacacagacaagcagggcaatctgaaagagctgtcttactaccaccatgataagtacaacatccaggagttcctgaagagacacgccaccatcaatgacaggattctggatacactgctgactgacgatctggacatctggaacttcaacttcgagaagttcgatttcgacaagaacgaggaaaaactgcagaatcaggaagataaggaccacattcaggctcatctgcaccatttcgtgtttgcagtcaataagatcaaaagcgagatggcatccggcgggcgccatcgaagccagtacttccaggaaatcaccaacgtgctggacgagaacaatcaccaggaaggctacctgaaaaacttctgtgagaatctgcataacaagaagtacagcaatctgtccgtgaagaatctggtcaacctgattggaaatctgtccaacctggaactgaagcccctgcgcaaatacttcaacgacaagatccacgctaaagcagaccattgggatgagcagaagtttactgaaacctattgccactggattctgggcgagtggcgggtgggggtcaaggatcaggacaagaaagacggcgcaaagtattcttacaaggacctgtgtaacgagctgaagcagaaagtgactaaggccgggctggtggacttcctgctggagctggacccctgccgaaccattccaccttacctggacaacaataacagaaagccacccaaatgtcagagcctgatcctgaatcccaagtttctggataatcagtatcctaactggcagcagtacctgcaggagctgaagaaactgcagtcaatccagaactacctggacagcttcgaaaccgatctgaaggtgctgaaaagctccaaggaccagccttacttcgtcgagtacaagtctagtaaccagcagatcgcttccggccagcgggattacaaggatctggacgcaagaatcctgcagttcatttttgacagggtgaaggcctctgatgagctgctgctgaacgaaatctatttccaggcaaagaaactgaagcagaaagcctcaagcgagctggaaaagctggagtcctctaagaaactggacgaagtgatcgctaactctcagctgagtcagattctgaagtctcagcacacaaatggaatcttcgagcagggcacttttctgcatctggtgtgcaaatactataagcagcgacagagagccagggacagccgcctgtacatcatgcctgaatatcgatacgataagaaactgcacaagtacaacaacaccggccgctttgacgatgacaaccagctgctgacatattgtaatcataagccccggcagaaaagataccagctgctgaacgacctggcaggagtgctgcaggtctctcctaattttctgaaggataaaatcgggtccgatgacgatctgttcatttctaagtggctggtggagcacatccggggctttaagaaggcctgcgaagacagcctgaaaatccagaaggataacaggggactgctgaatcataagatcaacattgcacgcaataccaagggcaaatgcgagaaagaaatcttcaacctgatctgtaagattgaggggagcgaagacaagaaagggaattataagcacggactggcctacgagctgggagtgctgctgttcggagagccaaacgaggccagcaagcccgaatttgataggaaaatcaagaaattcaattcaatctacagctttgcccagatccagcagattgcctttgctgagaggaaggggaatgcaaacacatgcgccgtgtgtagtgcagacaacgcccatcgcatgcagcagatcaaaattactgagccagtcgaagacaataaggataaaatcattctgtcagcaaaggcacagcgactgcctgcaatcccaacccgaattgtggatggagctgtcaagaaaatggctacaattctggcaaagaatatcgtggacgataattggcagaacattaagcaggtcctgagcgcaaaacaccagctgcatatcccaatcattaccgagtccaacgccttcgagtttgaacccgctctggcagacgtgaagggcaaatctctgaaggatagaaggaagaaagccctggagcgaattagtcccgaaaacatcttcaaggataagaacaacagaatcaaggagtttgctaaggggatttccgcctactctggagctaacctgacagatggggacttcgatggagcaaaggaggaactggatcacatcattcctcgcagccataagaaatatggcactctgaacgacgaggctaatctgatttgcgtgacccggggcgataataagaacaaagggaaccggatcttctgtctgagagacctggccgataattacaagctgaaacagtttgagaccacagacgatctggagatcgaaaagaaaattgccgacaccatctgggatgctaataagaaggacttcaagttcggaaactatcggagcttcatcaatctgacacctcaggagcagaaagcattcagacacgccctgtttctggctgatgaaaacccaatcaagcaggcagtgatcagagccattaataaccgcaaccgaaccttcgtgaatggcacacagaggtattttgctgaggtcctggcaaataacatctacctgcgcgccaagaaagaaaatctgaacactgacaagatcagcttcgattactttggaatccctaccattggaaacggccgagggatcgctgagattcggcagctgtatgaaaaggtggacagtgatatccaggcctacgctaaaggcgacaagccacaggcctcttatagtcacctgattgatgctatgctggcattctgcatcgccgctgacgagcatcggaacgatggatctattggcctggaaatcgacaaaaactatagtctgtaccctctggataagaatactggcgaggtgttcaccaaagacatcttttcacagatcaagattaccgacaacgagttcagcgataagaaactggtcagaaagaaagctattgaagggtttaacacacacagacagatgactagggatggaatctatgcagagaattacctgcctatcctgattcataaggagctgaacgaagtgaggaaggggtacacatggaaaaattccgaggaaatcaaaattttcaagggaaagaaatacgacatccagcagctgaataacctggtgtattgtctgaagtttgtggacaaaccaatcagtattgatatccagatttcaaccctggaggaactgagaaacatcctgactaccaataacattgcagccactgccgagtactattacattaatctgaaaacccagaagctgcacgagtattacatcgaaaattacaacacagccctggggtataagaaatacagcaaggagatggagttcctgaggtccctggcttataggtctgagcgcgtgaagatcaaaagtattgacgatgtcaagcaggtcctggacaaggattcaaacttcatcatcggaaagatcacactgcccttcaagaaagagtggcagcgactgtaccgggaatggcagaacacaactatcaaagacgattatgagtttctgaagagcttctttaatgtgaagtccattactaaactgcacaagaaagtccggaaagacttctctctgcccatcagtacaaacgagggcaagtttctggtgaagagaaaaacttgggataataacttcatctaccagattctgaatgactcagatagcagggcagacgggactaaacccttcattcctgcctttgatatcagcaagaacgagattgtggaagccatcattgacagtttcacctcaaaaaacatcttttggctgccaaagaatattgagctgcagaaggtggacaacaagaacatcttcgccattgataccagcaagtggtttgaggtcgaaacaccatccgacctgcgcgatatcggcattgctaccattcagtacaagatcgacaataactcacgccccaaggtgcgagtcaaactggattacgtgatcgacgatgacagcaagattaactatttcatgaatcactcactgctgaagagccggtatcccgacaaagtcctggagatcctgaagcagagcacaatcattgagttcgaaagttcagggtttaacaaaactattaaggagatgctgggaatgaagctggccggcatctacaatgaaacctccaataactaa

SEQ ID NO: 10 - DNA, 4890 bp, Francisella novicida
atgaattttaagatcctgcctatcgcaatcgacctgggcgtcaagaatactggcgtgtttagtgctttttatcagaaggggacctcactggagagactggacaataagaacggaaaagtgtatgaactgtccaaggattcttacactctgctgatgaacaataggaccgcacggagacaccagaggcgaggaattgacaggaaacagctggtgaagcgcctgttcaaactgatctggacagagcagctgaacctggaatgggataaggacactcagcaggccatcagcttcctgtttaatcgacggggattctcttttattactgacggctatagtcctgagtacctgaacatcgtgccagaacaggtcaaggcaatcctgatggacattttcgacgattataatggcgaggacgatctggattcctacctgaaactggccacagagcaagagagtaagatcagcgaaatctacaacaagctgatgcagaagatcctggagttcaagctgatgaaactgtgcaccgacatcaaggacgataaagtgagtaccaagacactgaaagagatcacaagctacgagttcgaactgctggccgattatctggctaactacagcgaatccctgaagacccagaaattttcctacacagacaagcagggcaatctgaaagagctgtcttactaccaccatgataagtacaacatccaggagttcctgaagagacacgccaccatcaatgacaggattctggatacactgctgactgacgatctggacatctggaacttcaacttcgagaagttcgatttcgacaagaacgaggaaaaactgcagaatcaggaagataaggaccacattcaggctcatctgcaccatttcgtgtttgcagtcaataagatcaaaagcgagatggcatccggcgggcgccatcgaagccagtacttccaggaaatcaccaacgtgctggacgagaacaatcaccaggaaggctacctgaaaaacttctgtgagaatctgcataacaagaagtacagcaatctgtccgtgaagaatctggtcaacctgattggaaatctgtccaacctggaactgaagcccctgcgcaaatacttcaacgacaagatccacgctaaagcagaccattgggatgagcagaagtttactgaaacctattgccactggattctgggcgagtggcgggtgggggtcaaggatcaggacaagaaagacggcgcaaagtattcttacaaggacctgtgtaacgagctgaagcagaaagtgactaaggccgggctggtggacttcctgctggagctggacccctgccgaaccattccaccttacctggacaacaataacagaaagccacccaaatgtcagagcctgatcctgaatcccaagtttctggataatcagtatcctaactggcagcagtacctgcaggagctgaagaaactgcagtcaatccagaactacctggacagcttcgaaaccgatctgaaggtgctgaaaagctccaaggaccagccttacttcgtcgagtacaagtctagtaaccagcagatcgcttccggccagcgggattacaaggatctggacgcaagaatcctgcagttcatttttgacagggtgaaggcctctgatgagctgctgctgaacgaaatctatttccaggcaaagaaactgaagcagaaagcctcaagcgagctggaaaagctggagtcctctaagaaactggacgaagtgatcgctaactctcagctgagtcagattctgaagtctcagcacacaaatggaatcttcgagcagggcacttttctgcatctggtgtgcaaatactataagcagcgacagagagccagggacagccgcctgtacatcatgcctgaatatcgatacgataagaaactgcacaagtacaacaacaccggccgctttgacgatgacaaccagctgctgacatattgtaatcataagccccggcagaaaagataccagctgctgaacgacctggcaggagtgctgcaggtctctcctaattttctgaaggataaaatcgggtccgatgacgatctgttcatttctaagtggctggtggagcacatccggggctttaagaaggcctgcgaagacagcctgaaaatccagaaggataacaggggactgctgaatcataagatcaacattgcacgcaataccaagggcaaatgcgagaaagaaatcttcaacctgatctgtaagattgaggggagcgaagacaagaaagggaattataagcacggactggcctacgagctgggagtgctgctgttcggagagccaaacgaggccagcaagcccgaatttgataggaaaatcaagaaattcaattcaatctacagctttgcccagatccagcagattgcctttgctgagaggaaggggaatgcaaacacatgcgccgtgtgtagtgcagacaacgcccatcgcatgcagcagatcaaaattactgagccagtcgaagacaataaggataaaatcattctgtcagcaaaggcacagcgactgcctgcaatcccaacccgaattgtggatggagctgtcaagaaaatggctacaattctggcaaagaatatcgtggacgataattggcagaacattaagcaggtcctgagcgcaaaacaccagctgcatatcccaatcattaccgagtccaacgccttcgagtttgaacccgctctggcagacgtgaagggcaaatctctgaaggatagaaggaagaaagccctggagcgaattagtcccgaaaacatcttcaaggataagaacaacagaatcaaggagtttgctaaggggatttccgcctactctggagctaacctgacagatggggacttcgatggagcaaaggaggaactggatcacatcattcctcgcagccataagaaatatggcactctgaacgacgaggctaatctgatttgcgtgacccggggcgataataagaacaaagggaaccggatcttctgtctgagagacctggccgataattacaagctgaaacagtttgagaccacagacgatctggagatcgaaaagaaaattgccgacaccatctgggatgctaataagaaggacttcaagttcggaaactatcggagcttcatcaatctgacacctcaggagcagaaagcattcagacacgccctgtttctggctgatgaaaacccaatcaagcaggcagtgatcagagccattaataaccgcaaccgaaccttcgtgaatggcacacagaggtattttgctgaggtcctggcaaataacatctacctgcgcgccaagaaagaaaatctgaacactgacaagatcagcttcgattactttggaatccctaccattggaaacggccgagggatcgctgagattcggcagctgtatgaaaaggtggacagtgatatccaggcctacgctaaaggcgacaagccacaggcctcttatagtcacctgattgatgctatgctggcattctgcatcgccgctgacgagcatcggaacgatggatctattggcctggaaatcgacaaaaactatagtctgtaccctctggataagaatactggcgaggtgttcaccaaagacatcttttcacagatcaagattaccgacaacgagttcagcgataagaaactggtcagaaagaaagctattgaagggtttaacacacacagacagatgactagggatggaatctatgcagagaattacctgcctatcctgattcataaggagctgaacgaagtgaggaaggggtacacatggaaaaattccgaggaaatcaaaattttcaagggaaagaaatacgacatccagcagctgaataacctggtgtattgtctgaagtttgtggacaaaccaatcagtattgatatccagatttcaaccctggaggaactgagaaacatcctgactaccaataacattgcagccactgccgagtactattacattaatctgaaaacccagaagctgcacgagtattacatcgaaaattacaacacagccctggggtataagaaatacagcaaggagatggagttcctgaggtccctggcttataggtctaggcgcgtgaagatcaaaagtattgacgatgtcaagcaggtcctggacaaggattcaaacttcatcatcggaaagatcacactgcccttcaagaaagagtggcagcgactgtaccgggaatggcagaacacaactatcaaagacgattatgagtttctgaagagcttctttaatgtgaagtccattactaaactgcacaagaaagtccggaaagacttctctctgcccatcagtacaaaccacggcaagtttctggtgaagagaaaaacttgggataataacttcatctaccagattctgaatgactcagatagcagggcagacgggactaaacccttcattcctgcctttgatatcagcaagaacgagattgtggaagccatcattgacagtttcacctcaaaaaacatcttttggctgccaaagaatattgagctgcagaaggtggacaacaagaacatcttcgccattgataccagcaagtggtttgaggtcgaaacaccatccgacctgcgcgatatcggcattgctaccattcagtacaagatcgacaataactcagcgcccaaggtgcgagtcaaactggattacgtgatcgacgatgacagcaagattaactatttcatgaatcactcactgctgaagagccggtatcccgacaaagtcctggagatcctgaagcagagcacaatcattgagttcgaaagttcagggtttaacaaaactattaaggagatgctgggaatgaagctggccggcatctacaatgaaacctccaataactaa

SEQ ID NO: 11 - RNA, 123 nt, Artificial Sequence
  Synthesized
gggaaauuaggugcgcuuggcguuucaguugcugaauuauuugguagaaauaccaaauaauuaaugcucuguaaucauuuaaaaguauuuugaacggaccucuguuugacacgucugaauaac

SEQ ID NO: 12 - DNA, 20 bp, Artificial Sequence
  Synthesized
ggaaattaggtgcgcttggc

SEQ ID NO: 13 - RNA, 20 nt, Artificial Sequence
  Synthesized
ugagcucccugacagcagcc

SEQ ID NO: 14 - RNA, 22 nt, Artificial Sequence
  Synthesized
ugagcucccugacagcagccac

SEQ ID NO: 15 - RNA, 24 nt, Artificial Sequence
  Synthesized
ugagcucccugacagcagccacac

SEQ ID NO: 16 - DNA, 20 bp, Artificial Sequence
  Synthesized
agaacaaagcccctgtgcta

SEQ ID NO: 17 - DNA, 20 bp, Artificial Sequence
  Synthesized
accactccaagcccttttct

SEQ ID NO: 18 - RNA, 22 nt, Artificial Sequence
  Synthesized
ccuagucuccaugagcucccug

SEQ ID NO: 19 - RNA, 22 nt, Artificial Sequence
  Synthesized
caccuuggggcaggaccaagug

SEQ ID NO: 20 - RNA, 22 nt, Artificial Sequence
  Synthesized
ugagcucccugacagcagccac

SEQ ID NO: 21 - RNA, 22 nt, Artificial Sequence
  Synthesized
gaguuccucaccuagucuccau

(19) United States
(12) Patent Application Publication
(10) Pub. No.: US 2018/0201912 A1
(43) Pub. Date: Jul. 19, 2018

(54) MODIFIED FNCAS9 PROTEIN AND USE THEREOF

(71) Applicant: The University of Tokyo, Tokyo (JP)

(72) Inventors: Osamu NUREKI, Tokyo (JP); Hiroshi NISHIMASU, Tokyo (JP); Hisato HIRANO, Tokyo (JP); Ryuichiro ISHITANI, Tokyo (JP)

(73) Assignee: The University of Tokyo, Tokyo (JP)

(21) Appl. No.: 15/743,419

(22) PCT Filed: Jul. 14, 2016
(86) PCT No.: PCT/JP2016/070815

§ 371 Date: Jan. 16, 2018

Related U.S. Application Data
(60) Provisional application No. 62/293,333, filed on Feb. 10, 2016.

(30) Foreign Application Priority Data
Jul. 14, 2015 (JP) 2015-140761

(51) Int. Cl. C12N 9/22; C12N 15/11; C12N 9/96; C12N 15/85; C12N 15/90

(52) U.S. Cl. CPC C12N 9/22; C12N 15/11; C12N 9/96; C12N 2800/80; C12N 15/907; C12N 2310/20; C12N 15/85

(57) ABSTRACT

The present invention provides a protein comprising a sequence containing any one of the amino acid sequences of the following (a) to (f) and having RNA-guided DNA endonuclease activity.

The present application claims priority on the basis of Japanese Patent Application No. 2015-140761 filed in Japan on Jul. 14, 2015, and on U.S. Patent No. 62/293,333 provisionally filed in the U.S. on Feb. 10, 2016, the contents of which are incorporated herein by reference.

### TECHNICAL FIELD

The present invention relates to a modified FnCas9 protein and the use thereof.

### BACKGROUND ART

Clustered regularly interspaced short palindromic repeats (CRISPR) are known to compose the adaptive immune system that provides acquired resistance against invasive exogenous nucleic acids in bacteria and archaea together with Cas (CRISPR-associated) genes. CRISPR frequently originate in phages or plasmid DNA and are composed of 24 bp to 48 bp short, conserved repeat sequences having characteristic variable DNA sequences referred to as spacers of similar size inserted there between. In addition, a group of genes encoding the Cas protein family is present in the vicinity of the repeat and spacer sequences.

In the CRISPR-Cas system, exogenous DNA is cleaved into roughly 30 bp fragments by members of the Cas protein family that are inserted into CRISPR. Members of the Cas protein family in the form of Cas1 and Cas2 proteins recognize a base sequence referred to as the proto-spacer motif (PAM) of exogenous DNA, excise a sequence upstream therefrom, and insert that sequence into the CRISPR sequence of the host to create an immunological memory of the bacteria. The CRISPR sequence containing immunological memory is then transcribed, and the resulting RNA (referred to as pre-crRNA) pairs with partially complementary RNA (trans-activating crRNA: tracrRNA) and is incorporated in another member of the Cas protein family in the form of Cas9 protein. The pre-crRNA and tracrRNA that have been incorporated in Cas9 are cleaved by RNase III resulting in small RNA fragments (CRISPR-RNAs:crRNAs) containing an exogenous sequence (guide sequence) and the formation a Cas9-crRNA-tracrRNA complex. The Cas9-crRNA-tracrRNA complex binds to exogenous invasive DNA complementary to crRNA, after which Cas9 protein, which is an enzyme that cleaves DNA (nuclease), cleaves the exogenous invasive DNA, and suppresses and eliminates the function of the DNA that has entered from the outside.

Cas9 protein recognizes a PAM sequence in exogenous invasive DNA and cleaves that DNA so as to blunten the ends of double-stranded DNA located upstream thereof. The lengths and base sequences of PAM sequences vary considerably, and in the case of Streptococcus pyogenes (S. pyogenes), for example, Cas9 protein recognizes the three-base sequence of “NGG”. Streptococcus thermophilus (S. thermophilus) has two Cas9 proteins that recognize PAM sequences in the form of sequences consisting of five or six bases in the form of “NGGNG” or “NNAGAA” (wherein, N represents an arbitrary base). Although the number of bp upstream from the PAM sequence where DNA is cleaved also varies according to the bacterial species, the majority of Cas9 orthologs, including that derived from S. pyogenes, cleaves at a location three bases upstream from the PAM sequence.

Technology has been actively developed in recent years for applying the CRISPR-Cas system in bacteria to genome editing. crRNA and tracrRNA are used by fusing and expressing as tracrRNA-crRNA chimera (to be referred to as guide RNA (gRNA)). As a result, nuclease (RNA-guided nuclease (RGN)) is then recruited to cleave genomic DNA at a target site.

Although the CRISPR-Cas system consists of types I, II and III, the type II CRISPR-Cas system is used nearly exclusively for genome editing, and Cas9 protein is used as RGN in type II systems. Since S. pyogenes-derived Cas9 protein recognizes the three-base sequence of NGG as a PAM sequence, DNA upstream therefrom can be cleaved as long as there is a sequence having two guanines in succession.

Methods using the CRISPR-Cas system enable genome editing using a single protein in the form of Cas9 protein simply by synthesizing a short gRNA sequence homologous to the target DNA sequence. Consequently, it is no longer necessary to synthesize large proteins differing for each DNA sequence in the manner of conventionally used zinc finger nucleases (ZFN) or transcription activator-like effector nucleases (TALEN), thereby enabling genome editing to be carried out easily and rapidly.

Patent Document 1 discloses a genome editing technology that uses a CRISPR-Cas system derived from S. pyogenes.

Patent Document 2 discloses a genome editing technology that uses a CRISPR-Cas system derived from S. thermophilus. Moreover, Patent Document 2 discloses that a Cas9 protein mutant D31A or N891A functions as a DNA nicking enzyme in the form of a nickase that places a nick only in one of the DNA strands. Moreover, these mutants are also indicated as having homologous recombination efficiency comparable to that of wild-type Cas9 protein while retaining a low incidence of non-homologous end-joining susceptible to the occurrence of insertions, deletions and other mutations in the repair mechanism following DNA cleavage.

Non-Patent Document 1 discloses a CRISPR-Cas system that uses S. pyogenes-derived Cas9, wherein the CRISPR-Cas system is a double nickase system that uses two Cas9 protein D10A mutants and a pair of target-specific guide RNA that form a complex with these D10A mutants. Each complex of Cas9 protein D10A mutant and target-specific guide RNA creates a nick in only one DNA strand homologous to the guide DNA. The pair of guide RNA has about 20 bases and only recognizes a target sequence located in the opposite strand of the target DNA. The two nicks created by each complex of Cas9 protein D10A mutant and target-specific guide RNA mimic a DNA double-strand break (DSB), and the use of the pair of guide RNA is indicated as being able to improve the specificity of Cas 9 protein-mediated genome editing while maintaining a high level of efficiency.

### PRIOR ART DOCUMENTS

### Patent Documents

Patent Document 1: International Publication No. 2014/093661
Patent Document 2: Japanese Translation of PCT International Application Publication No. 2015-510778

### Non-Patent Documents

Non-Patent Document 1: Ran, F. A., et al.: “Double Nicking by RNA-Guided CRISPR Cas9 for Enhanced Genome Editing Specificity”, Cell, Vol. 154, p. 1380-1389, 2013.

### DISCLOSURE OF THE INVENTION

### Problems to be Solved by the Invention

The PAM sequence able to be recognized by the Cas9 protein derived from S. pyogenes disclosed in Patent Document 1 consists of the three bases of “NGG”, while the PAM sequence able to be recognized by the Cas9 protein derived from S. thermophilus disclosed in Patent Document 2 consists of five or six bases of “NGGNG” or “NNAGAA”. Accordingly, since there are limitations on both of these recognizable PAM sequences, there are also limitations on the target sequences able to be edited using these sequences.

In addition, the double nickase system disclosed in Non-Patent Document 1 uses Cas9 protein derived from S. pyogenes, and since recognizable PAM sequences are required at a total of two locations in the sense strand and antisense strand within a target sequence, there are further limitations on those target sequences that are able to be edited.

With the foregoing in view, an object of the present invention is to provide a Cas9 protein that recognizes a wide range of PAM sequences while retaining binding strength with a target double-stranded polynucleotide and further retaining endonuclease activity. In addition, an object of the present invention is to provide a simple and rapid site-specific genome editing technology that uses the aforementioned Cas9 protein.

### Means for Solving the Problems

Namely, the present invention includes the aspects indicated below.

[1] A protein comprising a sequence containing any one of the amino acid sequences of the following (a) to (f) and having RNA-guided DNA endonuclease activity:

(a) amino acid sequence represented by SEQ ID NO: 1,

(b) amino acid sequence in which one or a plurality of amino acids have been deleted, inserted, substituted or added at sites other than amino acid positions 131, 211 and 318 of the amino acid sequence represented by SEQ ID NO: 1,

(c) amino acid sequence having identity of 80% or more at sites other than amino acid positions 131, 211 and 318 of the amino acid sequence represented by SEQ ID NO: 1,

(d) amino acid sequence represented by SEQ ID NO: 2,

(e) amino acid sequence in which one or a plurality of amino acids have been deleted, inserted, substituted or added at sites other than amino acid positions 1369, 1449 and 1556 of the amino acid sequence represented by SEQ ID NO: 2, and

(f) amino acid sequence having identity of 80% or more at sites other than amino acid positions 1369, 1449 and 1556 of the amino acid sequence represented by SEQ ID NO: 2.

[2] A gene encoding a protein comprising a sequence containing any one of the base sequences of the following (g) to (j) and having RNA-guided DNA endonuclease activity:

(g) base sequence represented by SEQ ID NO: 3 or 4,

(h) base sequence in which one or a plurality of bases have been deleted, substituted or added in the base sequence represented by SEQ ID NO: 3 or 4,

(i) base sequence having identity of 80% or more with the base sequence represented by SEQ ID NO: 1, and

(j) base sequence capable of hybridizing under stringent conditions with DNA composed of a sequence complementary to DNA composed of the base sequence represented by SEQ ID NO: 3 or 4.

[3] A protein-RNA complex provided with the protein described in [1] and a polynucleotide composed of a base sequence complementary to a base sequence located 1 to 20 to 24 bases upstream from a proto-spacer adjacent motif (PAM) sequence in a target double-stranded polynucleotide.

[4] A method for site-specifically cleaving a target double-stranded polynucleotide, including:

a step for mixing and incubating a target double-stranded polynucleotide, a protein and a guide RNA, and

a step for allowing the protein to cleave the target double-stranded polynucleotide at a cleavage site located 3 bases upstream from a PAM sequence to create blunt ends; wherein,

the target double-stranded polynucleotide has a PAM sequence composed of YG (wherein, Y represents a pyrimidine in the form of cytosine or thymine),

the protein is the protein described in [1], and

the guide RNA contains a polynucleotide composed of a base sequence complementary to a base sequence located 1 to 20 to 24 bases upstream from the PAM sequence in the target double-stranded polynucleotide.

[5] A method for site-specifically modifying a target double-stranded polynucleotide, including:

a step for mixing and incubating a target double-stranded polynucleotide, a protein and a guide RNA,

a step for allowing the protein to cleave the target double-stranded polynucleotide at a cleavage site located 3 bases upstream from a PAM sequence to create blunt ends, and

a step for obtaining a modified target double-stranded polynucleotide in a region determined by complementary binding between the guide RNA and the target double-stranded polynucleotide; wherein,

the target double-stranded polynucleotide has a PAM sequence composed of YG (wherein, Y represents a pyrimidine in the form of cytosine or thymine),

the protein is the protein described in [1], and

the guide RNA contains a polynucleotide composed of a base sequence complementary to a base sequence located 1 to 20 to 24 bases upstream from the PAM sequence in the target double-stranded polynucleotide.

[6] A method for selectively and site-specifically modifying a target double-stranded polynucleotide in a cell, including:

a step for injecting a protein A, a protein B and a guide RNA into a cell,

a step for restoring RNA-guided endonuclease activity by irradiating the cell with blue light to bind the protein A and the protein B,

a step for allowing the conjugate of the protein A and the protein B to cleave the target double-stranded polynucleotide at a cleavage site located 3 bases upstream from a PAM sequence to create blunt ends, and

a step for obtaining a modified target double-stranded polynucleotide in a region determined by complementary binding between the guide RNA and the target double-stranded polynucleotide; wherein,

the target double-stranded polynucleotide has a PAM sequence composed of YG (wherein, Y represents a pyrimidine in the form of cytosine or thymine),

the protein A is a protein having a photoswitching protein a bound to the C-terminal that comprises a protein composed of any of the amino acid sequences of the following (k) to (m) and has RNA-guided DNA endonuclease activity as a result of binding with the protein B:

(k) amino acid sequence represented by SEQ ID NO: 5,

(l) amino acid sequence in which one or a plurality of amino acids have been deleted, inserted, substituted or added in the amino acid sequence represented by SEQ ID NO: 5, and

(m) amino acid sequence having identity of 80% or more with the amino acid sequence represented by SEQ ID NO: 5,

the protein B is a protein having a photoswitching protein b bound to the N-terminal that comprises a protein composed of any of the amino acid sequences of the following (n) to (p) and has RNA-guided DNA endonuclease activity as a result of binding with the protein A:

(n) amino acid sequence represented by SEQ ID NO: 6,

(o) amino acid sequence in which one or a plurality of amino acids have been deleted, inserted, substituted or added at sites other than amino acid positions 526, 606 and 713 of the amino acid sequence represented by SEQ ID NO: 6, and

(p) amino acid sequence having identity of 80% or more at sites other than amino acid positions 526, 606 and 713 of the amino acid sequence represented by SEQ ID NO: 6, and

the guide RNA contains a polynucleotide composed of a base sequence complementary to a base sequence located 1 to 20 to 24 bases upstream from the PAM sequence in the target double-stranded polynucleotide.

[7] A method for producing target gene knockout cells using the method described in [6].

[8] A method for producing target gene knock-in cells using the method described in [6].

### Effects of the Invention

According to the present invention, a Cas9 protein can be obtained that recognizes a wide range of PAM sequences while retaining binding strength with a target double-stranded polynucleotide and further retaining endonuclease activity. In addition, a simple and rapid site-specific genome editing technology for a target sequence can be provided that uses the aforementioned Cas9 protein.

### BRIEF DESCRIPTION OF THE DRAWINGS

FIG. 1A is a drawing indicating recognizable PAM sequences in different species of bacteria.

FIG. 1B is a table indicating recognizable PAM sequences in different species of bacteria.

FIG. 2A is a drawing indicating the results of analyzing the crystal structure of a quaternary complex consisting of a Cas9 protein derived from F. novicida (FnCas9 protein), a guide RNA and a target double-stranded polynucleotide.

FIG. 2B is an enlarged view of FIG. 2A indicating the results of analyzing the crystal structure of a quaternary complex consisting of a Cas9 protein derived from F. novicida (FnCas9 protein), a guide RNA and a target double-stranded polynucleotide.

FIG. 3 is a drawing schematically representing examples of interaction and non-interaction between a PAM sequence recognition site and a target double-stranded polynucleotide in wild-type FnCas9 protein and Cas9 protein of the present embodiment.

FIG. 4A is a model diagram indicating interaction between glutamic acid at position 1449 in wild-type Cas9 protein and cytosine at position 2 in a sequence complementary to a PAM sequence.

FIG. 4B is a model diagram indicating interaction between histidine at position 1449 and cytosine at position 2 in a sequence complementary to a PAM sequence as an example of the Cas9 protein of the present embodiment.

FIG. 5A is a model diagram indicating interaction between arginine at position 1556 in a wild-type FnCas9 protein and guanine at position 2 in a PAM sequence.

FIG. 5B is a model diagram indicating non-interaction between alanine at position 1556 and guanine at position 2 in a PAM sequence as an example of the Cas9 protein of the present embodiment.

FIG. 6A is a model diagram indicating non-interaction between glutamic acid at position 1369 in a wild-type FnCas9 protein and adenine at position 1 and cytosine at position 2 in a sequence complementary to a PAM sequence.

FIG. 6B is a model diagram indicating interaction between arginine at position 1369 and a phosphate group located between adenine at position 1 and cytosine at position 2 in a sequence complementary to a PAM sequence.

FIG. 7 is a schematic diagram depicting cleavage of a target double-stranded polynucleotide by a Cas9 protein-guide RNA complex that recognizes a wide range of PAM sequences in the present embodiment.

FIG. 8 is a drawing indicating the steps of a method for site-specifically modifying a target double-stranded polynucleotide in a cell in the present embodiment.

FIG. 9 is a drawing for explaining cleavage of abase sequence in a target gene and the subsequent repair of the target gene in the present embodiment.

FIG. 10A is an image representing the results of agarose gel electrophoresis in a DNA cleavage activity measurement test in Example 1.

FIG. 10B is an image representing the results of agarose gel electrophoresis in a DNA cleavage activity measurement test in Example 1.

FIG. 11A is a graph indicating the developmental rates of embryos injected with various types of Cas9 and guide RNA in Example 2.

FIG. 11B depicts images indicating the morphology of blastocysts injected with FnCas9 and guide RNA of different lengths in Example 2.

FIG. 12 is a graph indicating knockout efficiency in blastocysts injected with various types of Cas9 and guide RNA in Example 2.

FIG. 13 is a graph indicating knockout efficiency in blastocysts injected with wild-type FnCas9 or mutant FnCas9 and guide RNA in Example 3.

### BEST MODE FOR CARRYING OUT THE INVENTION

The following provides a detailed explanation of embodiments of the present invention while referring to the drawings as necessary.

Furthermore, in the drawings, the same reference symbols are used to indicate identical or corresponding portions, and duplicate explanations thereof are omitted.

<Cas9 Protein Recognizing Wide Range of PAM Sequences>

In one embodiment thereof, the present invention provides a protein comprising a sequence containing any one of the amino acid sequences of the following (a) to (f) and having RNA-guided DNA endonuclease activity:

(a) amino acid sequence represented by SEQ ID NO: 1,

(b) amino acid sequence in which one or a plurality of amino acids have been deleted, inserted, substituted or added at sites other than amino acid positions 131, 211 and 318 of the amino acid sequence represented by SEQ ID NO: 1,

(c) amino acid sequence having identity of 80% or more at sites other than amino acid positions 131, 211 and 318 of the amino acid sequence represented by SEQ ID NO: 1,

(d) amino acid sequence represented by SEQ ID NO: 2,

(e) amino acid sequence in which one or a plurality of amino acids have been deleted, inserted, substituted or added at sites other than amino acid positions 1369, 1449 and 1556 of the amino acid sequence represented by SEQ ID NO: 2, and

(f) amino acid sequence having identity of 80% or more at sites other than amino acid positions 1369, 1449 and 1556 of the amino acid sequence represented by SEQ ID NO: 2.

The protein of the present embodiment is a Cas9 protein that recognizes a wide range of PAM sequences while retaining binding strength with a target double-stranded polynucleotide and further retaining endonuclease activity. According to the protein of the present embodiment, a simple and rapid technique can be provided for site-specific editing of the genome of a target sequence.

In the present description, the terms “polypeptide”, “peptide” and “protein” refer to polymers of amino acid residues and are used interchangeably. In addition, these terms also refer to amino acid polymers in which one or a plurality of amino acid residues are in the form of a chemical analog or modified derivative of the corresponding amino acids present in nature.

In the present description, a “sequence” refers to a nucleotide sequence of an arbitrary length, is a deoxyribonucleotide or ribonucleotide, and may be linear or branched and single-stranded or double-stranded.

In the present description, a “PAM sequence” refers to a sequence present in a target double-stranded polynucleotide that can be recognized by Cas9 protein, and differs according to the length of the PAM sequence while the base sequence differs according to the bacterial species. A sequence capable of being recognized by the Cas9 protein of the present invention capable of recognized a wide range of PAM sequences can be represented by “5′-YG-3′”

In the present description, a “polynucleotide” refers to a deoxyribonucleotide or ribonucleotide having linear or cyclic coordination and may be single-stranded or double-stranded, and should not be interpreted as being restricted with respect to polymer length. In addition, polynucleotides include known analogs of naturally-occurring nucleotides as well as nucleotides in which at least one of the base moieties, sugar moieties and phosphate moieties thereof has been modified (such as a phosphorothioate backbone). In general, an analog of a specific nucleotide has the same base-pairing specificity, and for example, A analogs form base pairs with T.

In the present description, “guide RNA” refers to that which mimics the hairpin structure of tracrRNA-crRNA, and contains in the 5′-terminal region thereof a polynucleotide composed of a base sequence complementary to a base sequence located from 1 to preferably 20 to 24 bases, and more preferably from 1 to preferably 22 to 24 bases, upstream from the PAM sequence in a target double-stranded polynucleotide. Moreover, guide RNA may contain one or more polynucleotides composed of a base sequence allowing the obtaining of a hairpin structure composed of base sequences non-complementary to a target double-stranded polynucleotide symmetrically arranged so as to form a complementary sequence having a single point as the axis thereof.

In order to obtain a Cas9 protein recognizing a wide range of PAM sequences, the inventors of the present invention investigated orthologs of Cas9 and focused on Cas9 protein derived from Francisella novicida (F. novicida), which is an ortholog of Cas9 that demonstrates the loosest restrictions on PAM recognition.

In the present invention, an “ortholog” refers to a group of genes that exhibit correspondence between genes derived accompanying species diversification from a common ancestral gene, or to a group of genes exhibiting such correspondence.

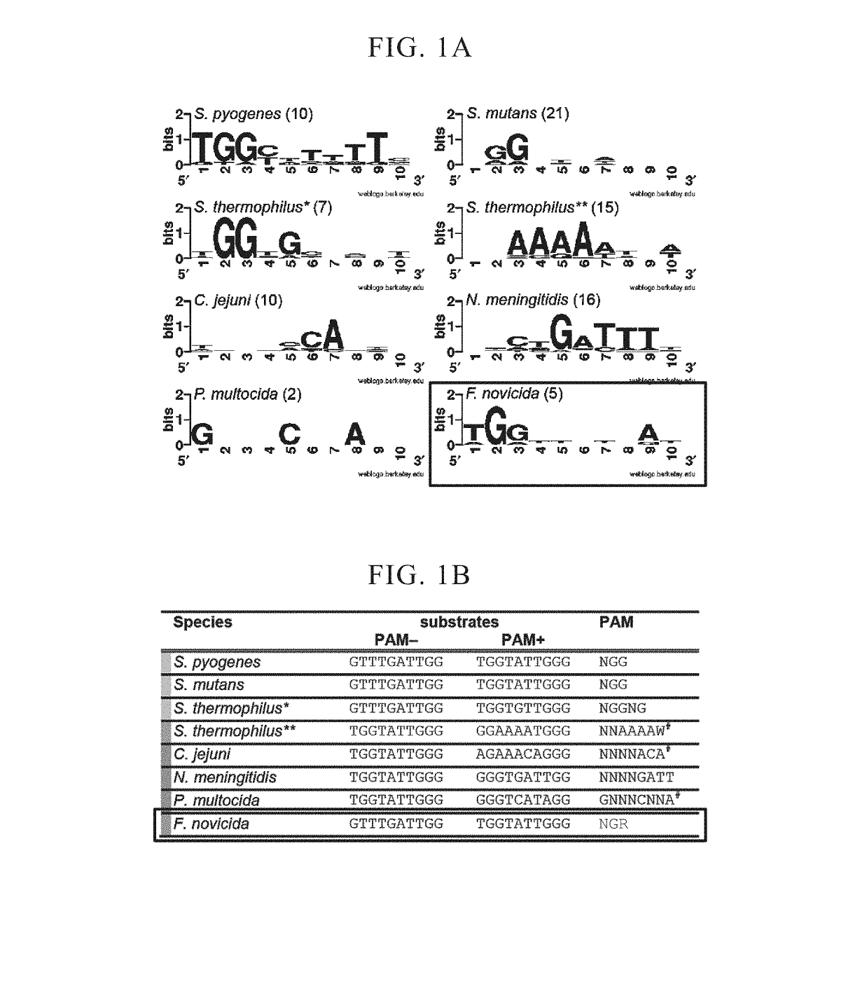

FIG. 1A is a drawing indicating recognizable PAM sequences in different species of bacteria, while FIG. 1B is a table indicating recognizable PAM sequences in different species of bacteria. The Cas9 protein derived from F. novicida (FnCas9 protein) is only required to recognize the three bases of 5′-NGR-3′, and can be understood to have looser restrictions attributable to the PAM sequence in comparison with the Cas9 proteins of other species.

Furthermore, in the present description, “N” refers to any one base selected from the group consisting of adenine, cytosine, thymine and guanine, “A” refers to adenine, “G” to guanine, “C” to cytosine, “T” to thymine, “R” to a base having a purine skeleton (adenine or guanine), and “Y” to a base having a pyrimidine skeleton (cytosine or thymine).

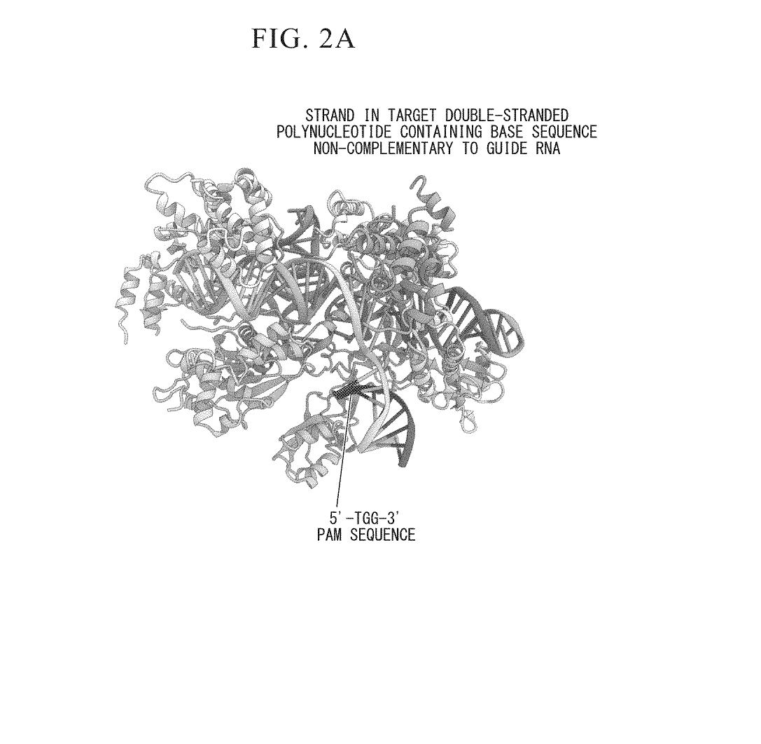

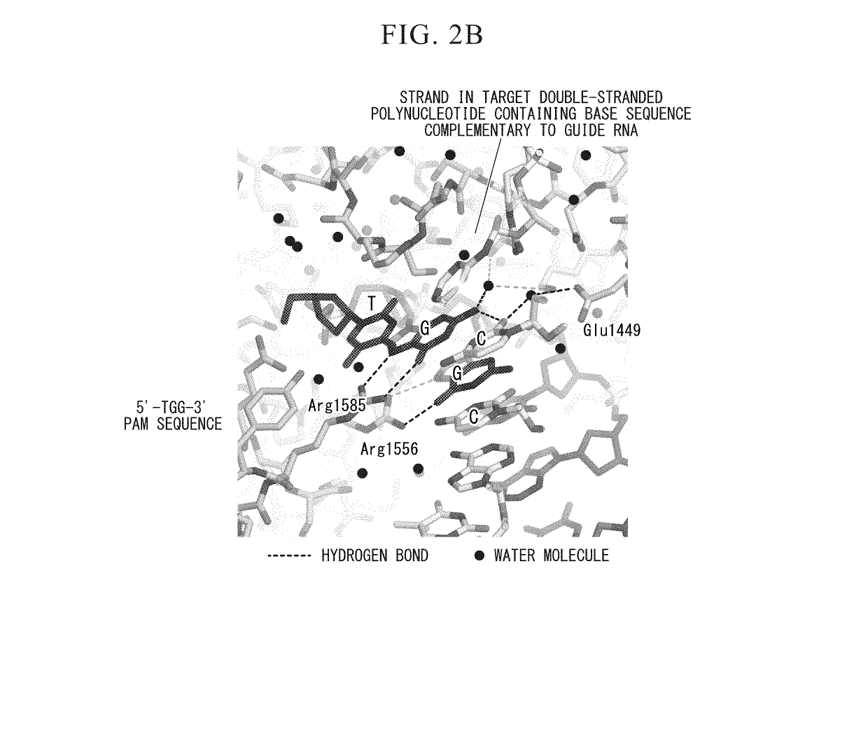

Continuing, the structure of the PAM recognition site was obtained by analyzing the crystal structure of a ternary complex of FnCas9 protein, guide RNA and a target double-stranded polynucleotide. FIGS. 2A and 2B are drawings indicating the results of analyzing the crystal structure of a quaternary complex consisting of FnCas9 protein, guide RNA and a target double-stranded polynucleotide. A target double-stranded polynucleotide having a strand containing “5′-TGG-3′” in a base sequence non-complementary to the guide RNA was used for the PAM sequence. It was determined from FIG. 2B that arginine at amino acid position 1585 of FnCas9 protein (Arg1585) and the guanine at the second position of the PAM sequence, as well as arginine at amino acid position 1556 of FnCas9 protein (Arg1556) and the guanine at the third position in the PAM sequence, form hydrogen bonds at the PAM sequence recognition site.

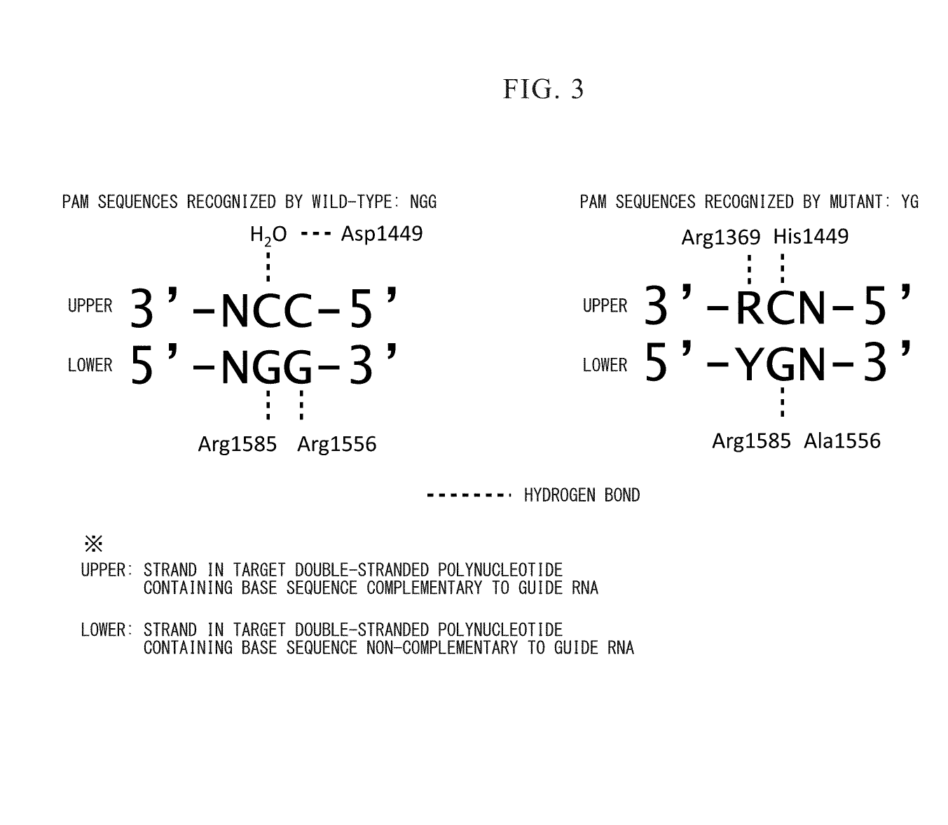

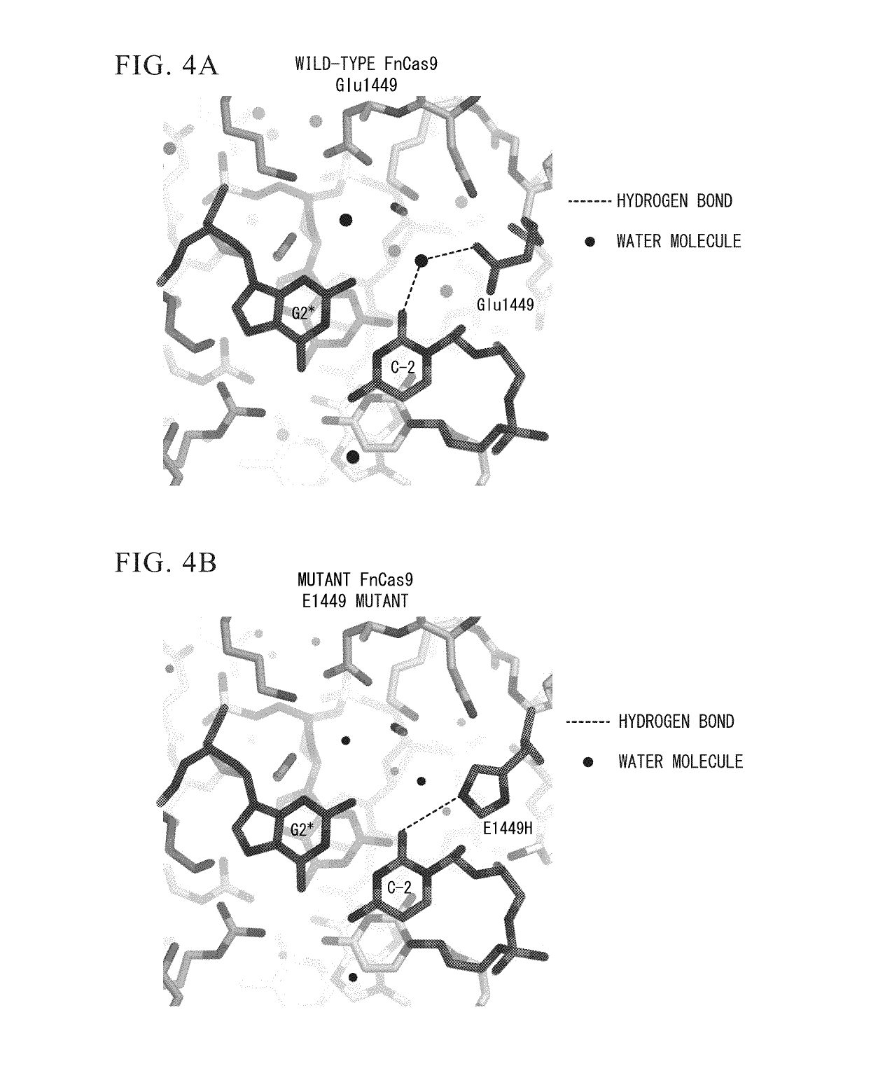

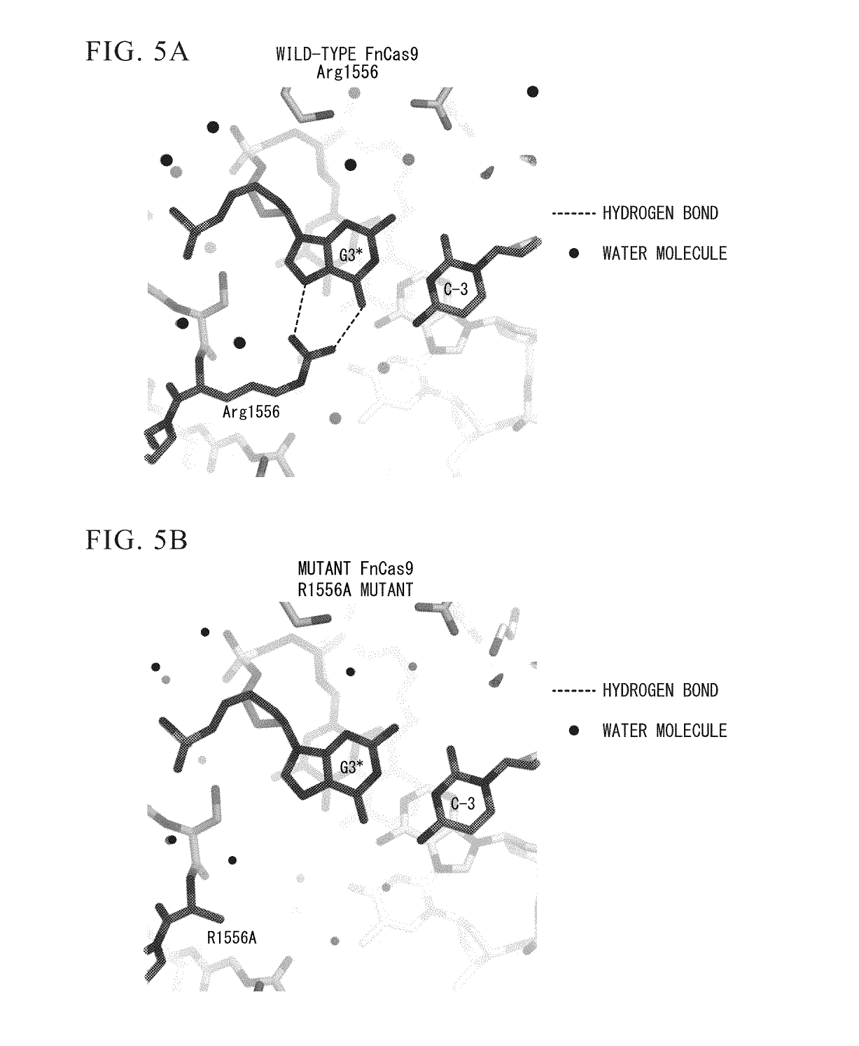

In addition, the left side of FIG. 3 is a drawing schematically representing interaction between a PAM recognition site and a target-double stranded polynucleotide in wild-type FnCa9 protein. In addition, FIGS. 4A, 5A and 6A are model diagrams indicating enlarged views of interaction between each amino acid at the PAM sequence recognition site and a target double-stranded polynucleotide in wild-type FnCas9 protein. In the “3′-NCC-5′” sequence complementary to the PAM sequence in the strand containing a base sequence complementary to the guide RNA in the target double-stranded polynucleotide, the arginine at position 1241 and the glutamic acid at position 1449 in the wild-type FnCas9 protein, and the cytosine at the second position in the sequence complementary to the PAM sequence, were determined to form hydrogen bonds through water molecules (refer to left side of FIG. 3 and FIG. 4A).

Therefore, attempts were made to modify the PAM sequence recognition site in the aforementioned wild-type FnCas9 protein, thereby leading to the invention of a Cas9 protein that recognizes a wide range of PAM sequences while retaining binding strength with a target double-stranded polynucleotide and further retaining endonuclease activity.

More specifically, the Cas9 protein recognizing a wide range of PAM sequences of the present invention is a protein composed of a sequence containing the amino acid sequence of the following (a) or (d):

(a) amino acid sequence represented by SEQ ID NO: 1, or

(d) amino acid sequence represented by SEQ ID NO: 2.

SEQ ID NO: 1 is the amino acid sequence of the PAM sequence recognition site in FnCas9 protein (consisting of 391 residues from methionine at position 1238 to asparagine at position 1629) that has been subjected to point mutation to as to recognize a wide range of PAM sequences.

SEQ ID NO: 2 is the full-length amino acid sequence of FnCas9 protein that has been subjected to point mutation so as to recognize a wide range of PAM sequences.

As a result of modifying aspartic acid at amino acid position 1449 of SEQ ID NO: 2 (amino acid position 211 of SEQ ID NO: 1) to an amino acid having a side chain capable of hydrogen-bonding with cytosine, binding strength can be enhanced since direct hydrogen bonding occurs between the PAM sequence in the target double-stranded polynucleotide and the cytosine at the second position in the sequence complementary to the PAM sequence (3′-N[C]C-5′). Examples of amino acids having a side chain capable of hydrogen-bonding with a nucleotide include asparagine, glutamine and histidine, and among these, histidine is preferable.

Moreover, as a result of modifying arginine at amino acid position 1556 of SEQ ID NO: 2 (amino acid position 318 of SEQ ID NO: 1) to an amino acid having a small molecular structure, a wider range of PAM sequences can be recognized since there is no longer hydrogen bonding with the guanine at the third position in the PAM sequence (5′-NG[G]-3′). Examples of amino acids having a small molecular structure include alanine, glycine, cysteine, isoleucine, leucine, methionine, proline, threonine, valine, asparagine, aspartic acid, glutamine and glutamic acid, and among these, alanine is preferable.

Moreover, as a result of modifying glutamic acid at amino acid position 1369 of SEQ ID NO: 2 (amino acid position 131 of SEQ ID NO: 1) to a basic amino acid or an amino acid capable of hydrogen-bonding with a phosphate residue in a base having a purine skeleton (adenine or guanine) of an arbitrary nucleic acid, bonding strength can be enhanced with a phosphate group in a base having a purine skeleton (adenine or guanine) in the arbitrary nucleic acid at the first position in the sequence complementary to the PAM sequence in the target double-stranded polynucleotide (3′-[N]CC-5′). Examples of basic amino acids include lysine and arginine. In addition, examples of amino acids capable of hydrogen-bonding with a phosphate group in a base having a purine skeleton (adenine or guanine) in an arbitrary nucleic acid include asparagine, glutamine and tyrosine. Among these, arginine is preferable.

The Cas9 protein recognizing a wide range of PAM sequences of the present invention comprises a protein composed of a sequence containing an amino acid sequence of the following (b), (c), (e) or (f) as a protein functionally equivalent to a protein composed of a sequence containing the amino acid sequence of the aforementioned (a) or (d):

(b) amino acid sequence in which one or a plurality of amino acids have been deleted, inserted, substituted or added at sites other than amino acid positions 131, 211 and 318 of the amino acid sequence represented by SEQ ID NO: 1,

(c) amino acid sequence having identity of 80% or more at sites other than amino acid positions 131, 211 and 318 of the amino acid sequence represented by SEQ ID NO: 1,

(e) amino acid sequence in which one or a plurality of amino acids have been deleted, inserted, substituted or added at sites other than amino acid positions 1369, 1449 and 1556 of the amino acid sequence represented by SEQ ID NO: 2, or

(f) amino acid sequence having identity of 80% or more at sites other than amino acid positions 1369, 1449 and 1556 of the amino acid sequence represented by SEQ ID NO: 2.

The amino acid sequence has identity of 80% or more in order to be functionally equivalent to the protein of the aforementioned (a) or (d). This identity is preferably 80% or more, more preferably 85% or more, even more preferably 90% or more, particularly preferably 95% or more, and most preferably 99% or more.

In addition, the number of amino acids that may be deleted, substituted or added here is preferably 1 to 15, more preferably 1 to 10, and particularly preferably 1 to 5.

In the present description, an “endonuclease” refers to an enzyme that cleaves a nucleotide strand at an intermediate location. Accordingly, the Cas9 protein that recognizes a wide range of PAM sequences of the present embodiment has enzyme activity guided by guide RNA that cleaves at an intermediate location of a DNA strand.

The protein of the present embodiment may be any protein composed of any one of the amino acid sequences of the aforementioned (a) to (f) provided it is a protein that has RNA-guided DNA endonuclease activity.

The right side of FIG. 3 is a drawing schematically representing an example of interaction between a PAM sequence recognition site and a target double-stranded polynucleotide in the Cas9 protein of the present embodiment. In addition, FIGS. 4B, 5B and 6B are model drawings indicating enlarged views of interaction and non-interaction between each amino acid at a modified PAM sequence recognition site, serving as an example of the Cas9 protein of the present embodiment, and a target double-stranded polynucleotide.

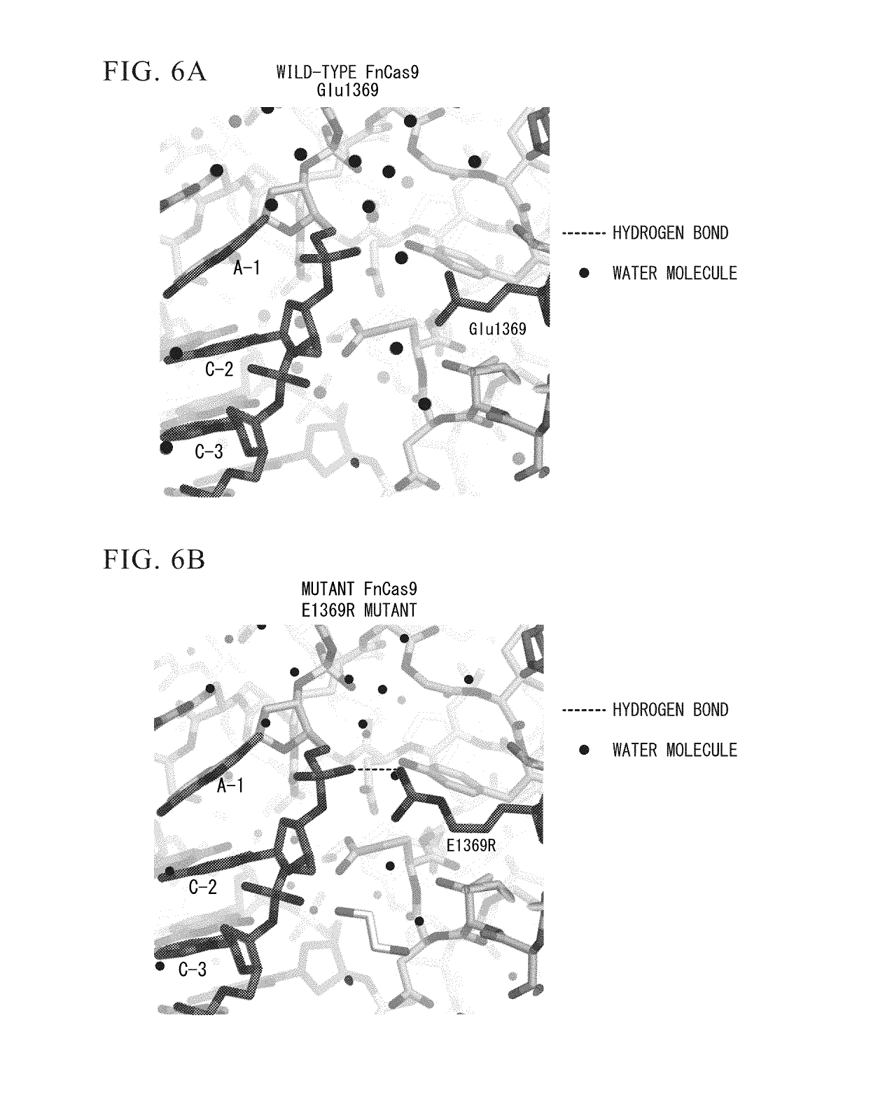

As indicated on the right side of FIG. 3 and in FIGS. 4B, 5B and 6B, the glutamic acid at position 1369 of the full length of FnCas9 protein is modified to the aspartic acid at position 1449, and the arginine at position 1556 is modified to alanine. The arginine at position 1369 of the modified FnCas9 protein enhances binding strength with the phosphate group in the base at the first position (adenine or guanine) having a purine skeleton (3′-[R]CC-5′) in the sequence complementary to the PAM sequence in the target double-stranded polynucleotide (refer to the right side of FIG. 3 and FIG. 6B). In addition, as a result of the histidine of position 1449 of the modified Cas9 protein hydrogen-bonding with cytosine at the second position (3′-R[C]C-5′) in the sequence complementary to the PAM sequence in the target double-stranded polynucleotide (refer to the right side of FIG. 3 and FIG. 4B), interaction is reinforced between the arginine at position 1585 of the modified Cas9 protein and the guanine at the second position in the PAM sequence (5′-Y[G]G-3′). Moreover, as a result of modifying the amino acid at position 1556 of the modified Cas9 protein to alanine, the recognizable PAM sequence becomes “5′-YG-3′” since there is no longer interaction with the guanine at the third position (5′-YG[G]-3′) in the PAM sequence, thereby enabling recognition of a wider range of PAM sequences.

The Cas9 protein recognizing a wide range of PAM sequences in the present embodiment can be produced according to, for example, the method indicated below. First, a host is transformed using a vector containing a nucleic acid that encodes the aforementioned Cas9 protein recognizing a wide range of PAM sequences. Continuing, the host is cultured to express the aforementioned protein. Conditions such as culture temperature, duration of culturing or addition of inducing agents can be determined by a person with ordinary skill in the art in accordance with known methods so that the transformant grows and the aforementioned protein is efficiently produced. In addition, in the case of having incorporated a selection marker in the form of an antibiotic resistance gene in an expression vector, the transformant can be selected by adding antibiotic to the medium. Continuing, Cas9 protein recognizing a wide range of PAM sequences is obtained by purifying the aforementioned protein expressed by the host according to a suitable method.

There are no particular limitations on the host, and examples thereof include animal cells, plant cells, insect cells and microorganisms such as Escherichia coli, Bacillus subtilis or yeast.

<Protein-Encoding Gene>

In one embodiment thereof, the present invention provides a gene encoding a protein comprising a sequence containing any one of the base sequences of the following (g) to (j) and having RNA-guided DNA endonuclease activity:

(g) base sequence represented by SEQ ID NO: 3 or 4,

(h) base sequence in which one or a plurality of bases have been deleted, substituted or added in the base sequence represented by SEQ ID NO: 3 or 4,

(i) base sequence having identity of 80% or more, preferably 85% or more, more preferably 90% or more and even more preferably 95% or more, with the base sequence represented by SEQ ID NO: 1, and

(j) base sequence capable of hybridizing under stringent conditions with DNA composed of a sequence complementary to DNA composed of the base sequence represented by SEQ ID NO: 3 or 4.

According to the gene of the present embodiment, Cas9 protein that recognizes a wide range of PAM sequences can be obtained while retaining binding strength with a target double-stranded polynucleotide and further retaining endonuclease activity.

SEQ ID NO: 3 is a base sequence of a gene that encodes a protein composed of the amino acid sequence of SEQ ID NO: 1. In addition, SEQ ID NO: 4 is a base sequence of a gene that encodes a protein composed of the amino acid sequence of SEQ ID NO: 2.

Here, the number of bases that may be deleted, substituted or added is preferably 1 to 30, more preferably 1 to 15, particularly preferably 1 to 10 and most preferably 1 to 5.

In the present description, an example of a method for hybridizing “under stringent conditions” is the method described in Molecular Cloning—A Laboratory Manual, Third Edition (Sambrooks, et al., Cold Spring Harbor Laboratory Press). Examples of such conditions include hybridizing by carrying out incubation for several hours to overnight at 55° C. to 70° C. in a hybridization buffer composed of 5×SSC (composition of 20×SSC: 3 M sodium chloride, 0.3 M citric acid solution, pH 7.0), 0.1% by weight N-lauroylsarcosine, 0.02% by weight SDS, 2% by weight hybridization blocking buffer and 50% formamide. Furthermore, the washing buffer used when washing the incubation solution is preferably a 0.1% by weight SDS-containing 1×SSC solution, and more preferably a 0.1% by weight SDS-containing 0.1×SSC

### Solution

<Complex of Cas9 Protein Recognizing Wide Range of PMA Sequences and Guide RNA>

In one embodiment thereof, the present invention provides a protein-RNA complex provided with the protein indicated in the previous section on <Cas9 Protein Recognizing Wide Range of PMA Sequences> and guide RNA containing a polynucleotide composed of a base sequence complementary to a base sequence located 1 to 20 to 24 bases upstream from a proto-spacer adjacent motif (PAM) sequence in a target double-stranded polynucleotide.

According to the protein-RNA complex of the present embodiment, a wide range of PMA sequences can be recognized and a target double-stranded polynucleotide can be easily and rapidly edited site-specifically for a target sequence.

The aforementioned protein and the aforementioned guide RNA are able to form a protein-RNA complex by mixing in vitro and in vivo under mild conditions. Mild conditions refer to a temperature and pH of a degree that does not cause protein decomposition or denaturation, and the temperature is preferably 4° C. to 40° C., while the pH is preferably 4 to 10.

In addition, the duration of mixing and incubating the aforementioned protein and the aforementioned guide RNA is preferably 0.5 hours to 1 hour. The complex formed by the aforementioned protein and the aforementioned guide RNA is able to maintain stability even if allowed to stand undisturbed for several hours at room temperature.

<CRISPR-Cas Vector System>

In one embodiment thereof, the present invention provides a CRISPR-Cas vector system provided with a first vector containing a gene encoding a protein indicated in the previous section on <Cas9 Protein Recognizing Wide Range of PAM Sequences>, and a second vector containing a guide RNA containing a polynucleotide composed of a base sequence complementary to a base sequence located 1 to 20 to 24 bases upstream from a proto-spacer adjacent motif (PAM) in a target double-stranded polynucleotide.

According to the CRISPR-Cas vector system of the present embodiment, a wide range of PMA sequences can be recognized and a target double-stranded polynucleotide can be easily and rapidly edited site-specifically for a target sequence.

An example of a gene encoding the protein indicated in the previous section on <Cas9 Protein Recognizing Wide Range of PAM Sequences> is the same as that exemplified in the previous section on <Protein-Encoding Gene>.

The guide RNA is suitably designed to contain in the 5′-terminal region thereof a polynucleotide composed of a base sequence complementary to a base sequence located from 1 to 20 to 24 bases, and preferably to 22 to 24 bases, upstream from a PAM sequence in a target double-stranded polynucleotide. Moreover, the guide RNA may also contain one or more polynucleotides composed of a base sequence allowing the obtaining of a hairpin structure composed of base sequences non-complementary to a target double-stranded polynucleotide symmetrically arranged so as to form a complementary sequence having a single point as the axis thereof.

The vector of the present embodiment is preferably an expression vector. There are no particular limitations on the expression vector, and examples thereof that can be used include E. coli-derived plasmids such as pBR322, pBR325, puC12 or puC13, B. subtilis-derived plasmids such as pUB110, pTP5 or pC194, yeast-derived plasmids such as pSH15, bacteriophages such as phages, viruses such as adenovirus, adeno-associated virus, lentivirus, vaccinia virus or baculovirus, and modified vectors thereof.

In the aforementioned expression vector, there are no particular limitations on the promoters for the aforementioned Cas9 protein or the aforementioned guide RNA, and examples thereof that can be used include promoters for expression in animal cells such as EF1α promoter, SRα promoter, SV40 promoter, LTR promoter or cytomegalovirus (CMV) promoter, promoters for expression in plant cells such as the 35S promoter of cauliflower mosaic virus (CaMV) or rubber elongation factor (REF) promoter, and promoters for expression in insect cells such as polyhedrin promoter or p10 promoter. These promoters can be suitably selected according to the aforementioned Cas9 protein and the aforementioned guide RNA, or the type of cells expressing the aforementioned Cas9 protein and the aforementioned guide RNA.

The aforementioned expression vector may also further have a multi-cloning site, enhancer, splicing signal, polyadenylation signal, selection marker or replication origin and the like.

<Method for Site-Specifically Cleaving Target Double-Stranded Polynucleotide>

### First Embodiment

In one embodiment thereof, the present invention provides a method for site-specifically cleaving a target double-stranded polynucleotide, provided with:

a step for mixing and incubating a target double-stranded polynucleotide, a protein and a guide RNA, and a step for allowing the protein to cleave the target double-stranded polynucleotide at a cleavage site located 3 bases upstream from a PAM sequence to create blunt ends; wherein,

the target double-stranded polynucleotide has a PAM sequence composed of YG (wherein, Y represents a pyrimidine in the form of cytosine or thymine),

the protein is the protein indicated in the previous section on <Cas9 Protein Recognizing Wide Range of PMA Sequences>, and

the guide RNA contains a polynucleotide composed of a base sequence complementary to a base sequence located 1 to 20 to 24 bases upstream from the PAM sequence in the target double-stranded polynucleotide.

According to the method of the present embodiment, a target double-stranded polynucleotide can be cleaved easily, rapidly and site-specifically for a target sequence by using RNA-guided DNA endonuclease recognizing a wide range of PAM sequences.

In the present embodiment, there are no particular limitations on the target double-stranded polynucleotide provided it has a PAM sequence composed of YG (wherein, Y represents a pyrimidine in the form of cytosine or thymine).

In the present embodiment, the protein and guide RNA are as indicated in the previous section on <Cas9 Protein Recognizing Wide Range of PMA Sequences>.

The following provides a detailed explanation of the method for site-specifically cleaving a target double-stranded polynucleotide.

First, the aforementioned protein and the aforementioned guide RNA are mixed and incubated under mild conditions. Mild conditions are as previously described. The incubation time is preferably 0.5 hours to 1 hour. A complex formed by the aforementioned protein and the aforementioned guide RNA is stable and is able to maintain stability even if allowed to stand for several hours at room temperature.

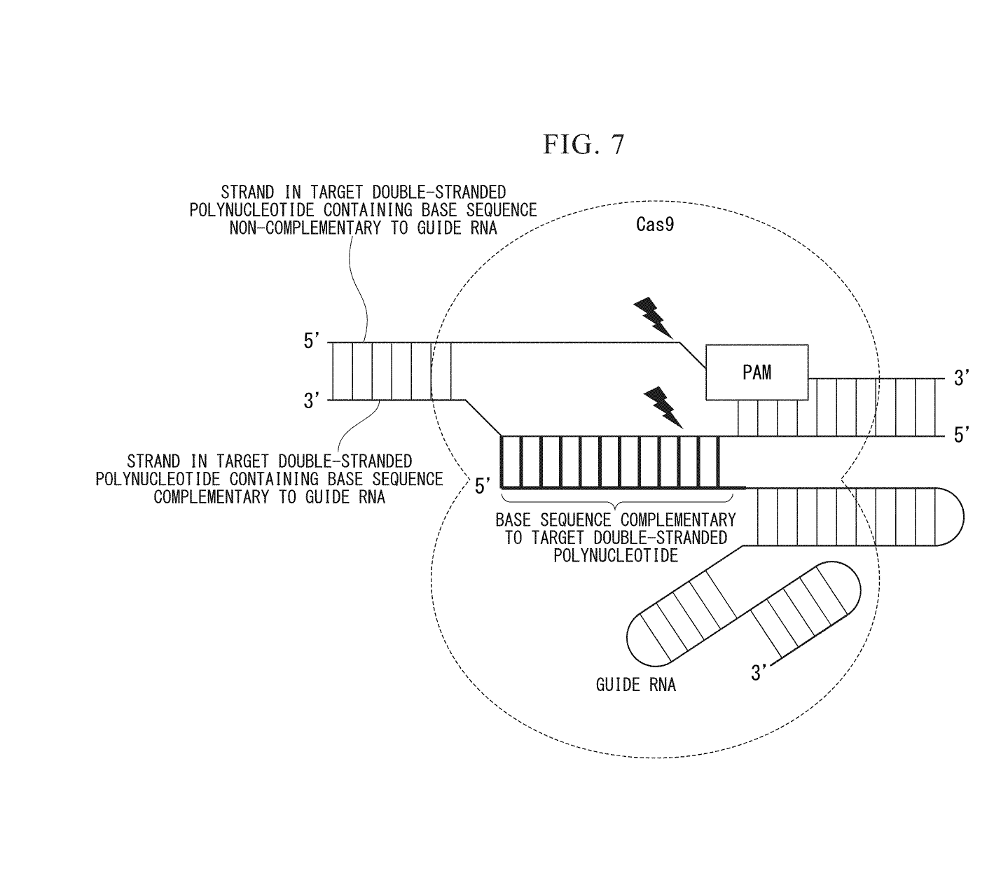

Next, the aforementioned protein and the aforementioned guide RNA form a complex in the aforementioned target double-stranded polynucleotide. The aforementioned protein recognizes PAM sequences composed of “5′-YG-3′”, and cleaves the target double-stranded polynucleotide at a cleavage site location three bases upstream from the PAM sequence to create blunt ends. FIG. 7 is a schematic diagram depicting cleavage of a target double-stranded polynucleotide by a Cas9 protein-guide RNA complex that recognizes a wide range of PAM sequences in the present embodiment. As a result of the Cas9 protein recognizing the PAM site, and the double helix structure of the target double-stranded polynucleotide being pulled apart starting at the PAM sequence and annealing with a base sequence complementary to the target double-stranded polynucleotide in the guide RNA, the double helix structure of the target double-stranded polynucleotide is partially unraveled. At this time, the aforementioned Cas9 protein cleaves phosphate ester bonds of the target double-stranded polynucleotide resulting in the creation of blunt ends at a cleavage site located three bases upstream from the PAM sequence and a cleavage site located three bases upstream from a sequence complementary to the PAM sequence.

### Second Embodiment

In the present embodiment, an expression step may be further provided prior to the incubation step in which the protein indicated in the previous section on <Cas9 Protein Recognizing Wide Range of PAM Sequences> and guide RNA are expressed using the previously described CRISPR-Cas vector system.

In the expression step of the present embodiment, Cas9 protein and guide RNA are first expressed using the aforementioned CRISPR-Cas vector system. A specific expression method consists of transforming a host using an expression vector containing a gene that encodes Cas9 protein and an expression vector containing guide RNA, respectively. Continuing, the host is cultured to express the Cas9 protein and guide RNA. Conditions such as culture temperature, duration of culturing or addition of inducing agents can be determined by a person with ordinary skill in the art in accordance with known methods so that the transformant grows and the aforementioned protein is efficiently produced. In addition, in the case of having incorporated a selection marker in the form of an antibiotic resistance gene in the expression vector, the transformant can be selected by adding antibiotic to the medium. Continuing, the Cas9 protein and guide RNA are obtained by purifying the Cas9 protein and guide RNA expressed by the host according to a suitable method.

<Method for Site-Specifically Modifying Target Double-Stranded Polynucleotide>

### First Embodiment

In one embodiment thereof, the present invention provides a method for site-specifically modifying a target double-stranded polynucleotide, provided with:

a step for mixing and incubating a target double-stranded polynucleotide, a protein and a guide RNA, a step for allowing the protein to cleave the target double-stranded polynucleotide at a cleavage site located 3 bases upstream from a PAM sequence to create blunt ends, and a step for obtaining a modified target double-stranded polynucleotide in a region determined by complementary binding between the guide RNA and the target double-stranded polynucleotide; wherein,

the target double-stranded polynucleotide has a PAM sequence composed of YG (wherein, Y represents a pyrimidine in the form of cytosine or thymine),

the protein is the protein indicated in the previous section on <Cas9 Protein Recognizing Wide Range of PAM Sequences>, and

the guide RNA contains a polynucleotide composed of a base sequence complementary to a base sequence located 1 to 20 to 24 bases upstream from the PAM sequence in the target double-stranded polynucleotide.

According to the method of the present embodiment, a target double-stranded polynucleotide can be modified easily, rapidly and site-specifically for a target sequence by using RNA-guided DNA endonuclease recognizing a wide range of PAM sequences.

In the present embodiment, the target double-stranded polynucleotide, protein and guide RNA are as indicated in the previous sections on <Cas9 Protein Recognizing Wide Range of PMA Sequences> and <Method for Site-Specifically Cleaving Target Double-Stranded Polynucleotide>.

The following provides a detailed explanation of the method for site-specifically modifying a target double-stranded polynucleotide. First, the steps through site-specifically cleaving a target double-stranded polynucleotide are the same as in the previous section on <Method for Site-Specifically Cleaving Target Double-Stranded Polynucleotide>. Continuing, a target double-stranded polynucleotide that has been modified as necessary is obtained in a region determined by complementary binding between the guide RNA and the target double-stranded polynucleotide.

In the present description, “modification” refers to a change in the base sequence of a target double-stranded polynucleotide. Examples thereof include cleavage of a target double-stranded polynucleotide, alteration of the base sequence of a target double-stranded polynucleotide by inserting an exogenous sequence following cleavage (by physical insertion or insertion by replicating through homology-directed repair), and alteration of the base sequence of a target double-stranded polynucleotide by non-homologous end-joining (NHEJ: rejoining the ends of DNA resulting from cleavage) following cleavage.

Modification of a target double-stranded polynucleotide in the present embodiment makes it possible to introduce a mutation into the target double-stranded polynucleotide or disrupt the function of the target double-stranded polynucleotide.

### Second Embodiment

In the present embodiment, an expression step may be further provided prior to the incubation step in which the protein indicated in the previous section on <Cas9 Protein Recognizing Wide Range of PAM Sequences> and guide RNA are expressed using the previously described CRISPR-Cas vector system.

In the expression step of the present embodiment, Cas9 protein and guide RNA are first expressed using the aforementioned CRISPR-Cas vector system. The specific expression method is similar to the method exemplified in the second embodiment in the previous section on <Method for Site-Specifically Cleaving Target Double-Stranded Polynucleotide>.

<Method for Site-Specifically Modifying Target Double-Stranded Polynucleotide in Cells>

In one embodiment thereof, the present invention provides a method for site-specifically modifying a target double-stranded polynucleotide in cells, provided with:

a step for introducing the previously described CRISPR-Cas9 vector system into a cell and expressing protein indicated in the previous section on <Cas9 Protein Recognizing Wide Range of PAM Sequences> and guide RNA,

a step for allowing the protein to cleave the target double-stranded polynucleotide at a cleavage site located 3 bases upstream from a PAM sequence to create blunt ends, and

a step for obtaining a modified target double-stranded polynucleotide in a region determined by complementary binding between the guide RNA and the target double-stranded polynucleotide; wherein,

the target double-stranded polynucleotide has a PAM sequence composed of YG (wherein, Y represents a pyrimidine in the form of cytosine or thymine), and

the guide RNA contains a polynucleotide composed of a base sequence complementary to a base sequence located 1 to 20 to 24 bases upstream from the PAM sequence in the target double-stranded polynucleotide.

In the expression step of the present embodiment, Cas9 protein and guide RNA are expressed in a cell using the aforementioned CRISPR-Cas vector system.

Examples of organisms serving as the origin of the cells targeted for application of the method of the present embodiment include prokaryotes, yeasts, animals, plants and insects. There are no particular limitations on the aforementioned animals, and examples thereof include, but are not limited to, humans, monkeys, dogs, cats, rabbits, pigs, cows, mice and rats.

In addition, the type of organism serving as the source of the cells can be arbitrarily selected according to the desired type or objective of the target double-stranded polynucleotide.

Examples of animal-derived cells targeted for application of the method of the present embodiment include, but are not limited to, germ cells (such as sperm or ova), somatic cells composing the body, stem cells, progenitor cells, cancer cells isolated from the body, cells isolated from the body that are stably maintained outside the body as a result of having become immortalized, and cells isolated from the body for which the nuclei have been artificially replaced.

Examples of somatic cells composing the body include, but are not limited to, cells harvested from arbitrary tissue such as the skin, kidneys, spleen, adrenals, liver, lungs, ovaries, pancreas, uterus, stomach, small intestine, large intestine, urinary bladder, prostate gland, testes, thymus, muscle, connective tissue, bone, cartilage, vascular tissue, blood, heart, eyes, brain or neural tissue. Specific examples of somatic cells include, but are not limited to, fibroblasts, bone marrow cells, immune cells (such as B lymphocytes, T lymphocytes, neutrophils, macrophages or monocytes), erythrocytes, platelets, osteocytes, bone marrow cells, pericytes, dendritic cells, keratinocytes, adipocytes, mesenchymal cells, epithelial cells, epidermal cells, endothelial cells, intravascular endothelial cells, lymphatic endothelial cells, hepatocytes, pancreatic islet cells (such as α cells, β cells, δ cells, ε cells or PP cells), chondrocytes, cumulus cells, glia cells, nerve cells (neurons), oligodendrocytes, microglia cells, astrocytes, cardiomyocytes, esophageal cells, muscle cells (such as smooth muscle cells or skeletal muscle cells), melanocytes and mononuclear cells.

Stem cells refer to cells having both the ability to self-replicate as well as the ability to differentiate into a plurality of other cell lines. Examples of stem cells include, but are not limited to, embryonic stem cells (ES cells), embryonic tumor cells, embryonic germ stem cells, induced pluripotent stem cells (iPS cells), neural stem cells, hematopoietic stem cells, mesenchymal stem cells, hepatic stem cells, pancreatic stem cells, muscle stem cells, germ stem cells, intestinal stem cells, cancer stem cells and hair follicle stem cells.

Cancer cells are cells derived from somatic cells that have acquired reproductive integrity. Examples of the origins of cancer cells include, but are not limited to, breast cancer (such as invasive ductal carcinoma, non-invasive ductal carcinoma or inflammatory breast cancer), prostate cancer (such as hormone-dependent prostate cancer or hormone-independent prostate cancer), pancreatic cancer (such as pancreatic ductal carcinoma), gastric cancer (such as papillary adenocarcinoma, mucinous carcinoma or adenosquamous carcinoma), lung cancer (such as non-small cell lung cancer, small cell lung cancer or malignant mesothelioma), colon cancer (such as gastrointestinal stromal tumor), rectal cancer (such as gastrointestinal stromal tumor), colorectal cancer (such as familial colorectal cancer, hereditary non-polyposis colon cancer or gastrointestinal stromal tumor), small intestine cancer (such as non-Hodgkin's lymphoma or gastrointestinal stromal tumor), esophageal cancer, duodenal cancer, tongue cancer, pharyngeal cancer (such as nasopharyngeal carcinoma, oropharyngeal carcinoma or hypopharyngeal carcinoma), head and neck cancer, salivary gland cancer, brain tumor (such as pineal astrocytoma, pilocytic astrocytoma, diffuse astrocytoma or anaplastic astrocytoma), schwannoma, liver cancer (such as primary liver cancer or extrahepatic bile duct cancer), kidney cancer (such as renal cell carcinoma or transitional cell carcinoma of the renal pelvis and ureter), gallbladder cancer, bile duct cancer, pancreatic cancer, endometrial carcinoma, cervical cancer, ovarian cancer (such as epithelial ovarian cancer, extragonadal germ cell tumor, ovarian germ cell tumor or ovarian low malignant potential tumor), bladder cancer, urethral cancer, skin cancer (such as intraocular (ocular) melanoma or Merkel cell carcinoma), hemangioma, malignant lymphoma (such as reticulum cell sarcoma, lymphosarcoma or Hodgkin's disease), melanoma (such as malignant melanoma), thyroid cancer (such as medullary thyroid cancer), parathyroid cancer, nasal cancer, paranasal cancer, bone tumor (such as osteosarcoma, Ewing's tumor, uterine sarcoma or soft tissue sarcoma), metastatic medulloblastoma, angiofibroma, protuberant dermatofibrosarcoma, retinal sarcoma, penile cancer, testicular tumor, pediatric solid tumor (such as Wilms tumor or pediatric kidney tumor), Kaposi's sarcoma, AIDS-induced Kaposi's sarcoma, maxillary sinus tumor, fibrous histiocytoma, leiomyosarcoma, rhabdomyosarcoma, chronic myeloproliferative disease and leukemia (such as acute myelogenous leukemia or acute lymphoblastic leukemia).

Cell lines refer to cells that have acquired reproductive integrity through artificial manipulation in vitro. Examples of cell lines include, but are not limited to, HCT116, Huh7, HEK293 (human embryonic kidney cells), HeLa (human cervical cancer cell line), HepG2 (human liver cancer cell line), UT7/TPO (human leukemia cell line), CHO (Chinese hamster ovary cell line), MDCK, MDBK, BHK, C-33A, HT-29, AE-1, 3D9, NsO/1, Jurkat, NIH3T3, PC12, S2, Sf9, Sf21, High Five and Vero.

Introduction of the CRISPR-Cas vector system into cells can be carried out using a method suitable for the viable cells used, and examples thereof include electroporation, heat shock method, calcium phosphate method, lipofection, DEAE dextran method, microinjection, particle gun method, methods using viruses, and methods using commercially available transfection reagents such as FuGENE® 6 Transfection Reagent (Roche Diagnostics GmbH), Lipofectamine 2000 Reagent (Invitrogen Corp.), Lipofectamine LTX Reagent (Invitrogen Corp.) or Lipofectamine 3000 Reagent (Invitrogen Corp.).

Continuing, the blunt end creation step and modification step are the same as the methods indicated in the first embodiment in the previous section on <Method for Site-Specifically Modifying Target Double-Stranded Polynucleotide>.

Modification of a target double-stranded polynucleotide in the present embodiment makes it possible to obtain cells in which a mutation has been introduced into the target double-stranded polynucleotide or the function of the target double-stranded polynucleotide has been disrupted.

<Method for Selectively and Site-Specifically Modifying Target Double-Stranded Polynucleotide in Cells>

In one embodiment thereof, the present invention provides a method for selectively and site-specifically modifying a target double-stranded polynucleotide in a cell, provided with:

a step for injecting a protein A, a protein B and a guide RNA into a cell,

a step for restoring RNA-guided endonuclease activity by irradiating the cell with blue light to bind the protein A and the protein B,

a step for allowing the conjugate of the protein A and the protein B to cleave the target double-stranded polynucleotide at a cleavage site located three bases upstream from a PAM sequence to create blunt ends, and

a step for obtaining a modified target double-stranded polynucleotide in a region determined by complementary binding between the guide RNA and the target double-stranded polynucleotide; wherein,

the target double-stranded polynucleotide has a PAM sequence composed of YG (wherein, Y represents a pyrimidine in the form of cytosine or thymine),

the protein A is a protein having a photoswitching protein a bound to the C-terminal that comprises a protein containing any of the amino acid sequences of the following (k) to (m) and having RNA-guided DNA endonuclease activity as a result of binding with the protein B:

(k) amino acid sequence represented by SEQ ID NO: 5,

(l) amino acid sequence in which one or a plurality of amino acids have been deleted, inserted, substituted or added in the amino acid sequence represented by SEQ ID NO: 5, and

(m) amino acid sequence having identity of 80% or more with the amino acid sequence represented by SEQ ID NO: 5, the protein B is a protein having a photoswitching protein b bound to the N-terminal that comprises a protein containing any of the amino acid sequences of the following (n) to (p) and having RNA-guided DNA endonuclease activity as a result of binding with the protein A:

(n) amino acid sequence represented by SEQ ID NO: 6,

(o) amino acid sequence in which one or a plurality of amino acids have been deleted, inserted, substituted or added at sites other than amino acid positions 526, 606 and 713 of the amino acid sequence represented by SEQ ID NO: 6, and

(p) amino acid sequence having identity of 80% or more at sites other than amino acid positions 526, 606 and 713 of the amino acid sequence represented by SEQ ID NO: 6, and

the guide RNA contains a polynucleotide composed of a base sequence complementary to a base sequence located 1 to 20 to 24 bases upstream from the PAM sequence in the target double-stranded polynucleotide.

According to the method of the present embodiment, the use of RNA-guided DNA endonuclease recognizing a wide range of PAM sequences makes it possible to easily, rapidly and site-specifically modify a target double-stranded polynucleotide for a target sequence.

Although the Cas9 protein recognizing a wide range of PAM sequences of the present embodiment relaxes the restrictions on target selection as a result of recognizing a wider range of PAM sequences, there is the risk of an increase in off-target effects caused by a decrease in specificity attributable to the PAM sequence. Accordingly, the inventors of the present invention found that Cas9 activity can be controlled by dividing the Cas protein into two moieties, and using a fusion protein obtained by binding a photoswitching protein to the C-terminal of a protein composed of the amino acid residues on the N-terminal side of Cas protein, and using a fusion protein obtained by binding a photoswitching protein to the N-terminal of a protein composed of the amino acid residues on the C-terminal side of Cas protein, thereby leading to completion of the present invention.

In the present description, a “photoswitching protein” refers to a pair of proteins obtained by performing multilateral protein engineering on a small VIVID photoreceptor possessed by red bread mold (Neurospora crassa) developed by the research group of Moritoshi Satoh of the Tokyo University Graduate School of Arts and Sciences (Nat. Commun. 6, 6256 (2015).doi: 10. 1038/ncomms7256). The pair of photoswitching proteins exists as a monomer in dark locations and forms a heterodimer when exposed to blue light. Various light-activated tools can be designed and developed using this conversion between monomer and dimer triggered by light. The amino acid sequence of photoswitching protein a is shown in SEQ ID NO: 7, while the amino acid sequence of photoswitching protein b is shown in SEQ ID NO: 8.

Examples of the cells targeted for application of the method of the present embodiment are the same as those exemplified in the previous section on <Method for Site-Specifically Modifying Target Double-Stranded Polynucleotide in Cells>.

In addition, examples of organisms serving as the origins of the cells include prokaryotes, yeasts, animals, plants and insects. Examples of the aforementioned animals include, but are not limited to, humans, monkeys, dogs, cats, rabbits, pigs, cows, mice and rats.

In addition, the type of organism serving as the source of the cells can be arbitrarily selected according to the desired type or objective of the target double-stranded polynucleotide.

[Protein A]

More specifically, protein A of the present embodiment is a protein having a photoswitching protein a bound to the C-terminal thereof that comprises a protein composed of the amino acid sequence of the following (k) or (n) and has RNA-guided DNA endonuclease activity as a result of binding with the aforementioned protein B:

(k) amino acid sequence represented by SEQ ID NO: 5.

SEQ ID NO: 5 is an amino acid sequence consisting of 829 residues from amino acid position 1 to amino acid position 829 on the N-terminal side of SEQ ID NO: 2.

In addition, photoswitching protein a is preferably bound to protein A through a flexible linker composed of a total of 16 amino acid residues consisting of eight repeats of the two bases of Gly-Ser.

More specifically, protein A of the present embodiment is a fusion protein having photoswitching protein a bound to the C-terminal thereof that comprises a protein composed of the amino acid sequence of the following (1) or (m) that is functionally equivalent to a protein composed of the amino acid sequence of the aforementioned (k):

(l) amino acid sequence in which one or a plurality of amino acids have been deleted, inserted, substituted or added in the amino acid sequence represented by SEQ ID NO: 5, or

(m) amino acid sequence having identity of 80% or more with the amino acid sequence represented by SEQ ID NO: 5.

The amino acid sequence has identity of 80% or more in order to be functionally equivalent to the protein composed of the amino acid sequence of the aforementioned (k). This identity is preferably 80% or more, more preferably 85% or more, even more preferably 90% or more, particularly preferably 95% or more, and most preferably 99% or more.

In addition, the number of amino acids that may be deleted, substituted or added here is preferably 1 to 15, more preferably 1 to 10, and particularly preferably 1 to 5.

[Protein B]

More specifically, the protein B of the present embodiment is a protein having a photoswitching protein b bound to the N-terminal thereof that comprises a protein composed of the amino acid sequence of the following (n) and has RNA-guided DNA endonuclease activity as a result of binding with the aforementioned protein A:

(n) amino acid sequence represented by SEQ ID NO: 6.

SEQ ID NO: 6 is an amino acid sequence consisting of 786 residues from amino acid position 844 to amino acid position 1629 on the C-terminal side of SEQ ID NO: 2.

In addition, photoswitching protein b is preferably bound to protein B through a flexible linker composed of a total of 16 amino acid residues consisting of eight repeats of the two bases of Gly-Ser.

More specifically, protein B of the present embodiment is a fusion protein having photoswitching protein b bound to the N-terminal thereof that comprises a protein composed of the amino acid sequence of the following (o) or (p) that is functionally equivalent to a protein composed of the amino acid sequence of the aforementioned (n):

(o) amino acid sequence in which one or a plurality of amino acids have been deleted, inserted, substituted or added at sites other than amino acid positions 526, 606 and 713 of the amino acid sequence represented by SEQ ID NO: 6, or

(p) amino acid sequence having identity of 80% or more with sites other than amino acid positions 526, 606 and 713 of the amino acid sequence represented by SEQ ID NO: 6.

The amino acid sequence has identity of 80% or more in order to be functionally equivalent to the protein composed of the amino acid sequence of the aforementioned (n). This identity is preferably 80% or more, more preferably 85% or more, even more preferably 90% or more, particularly preferably 95% or more, and most preferably 99% or more.

In addition, the number of amino acids that may be deleted, substituted or added here is preferably 1 to 15, more preferably 1 to 10, and particularly preferably 1 to 5.

In the present embodiment, the aforementioned protein A may be a protein composed only of any one of the amino acid sequences of the aforementioned (k) to (m) provided it is a fusion protein having the photoswitching protein a bound to the C-terminal thereof and has RNA-guided DNA endonuclease activity as a result of binding with the aforementioned protein B.

Similarly, the aforementioned protein B may be a protein composed only of any one of the amino acid sequences of the aforementioned (n) to (p) provided it is a fusion protein having the photoswitching protein b bound to the N-terminal thereof and has RNA-guided DNA endonuclease activity as a result of binding with the aforementioned protein A.

The aforementioned protein A and the aforementioned protein B can be produced according to the method indicated in the previous section on <Cas9 Protein Recognizing Wide Range of PAM Sequences>.

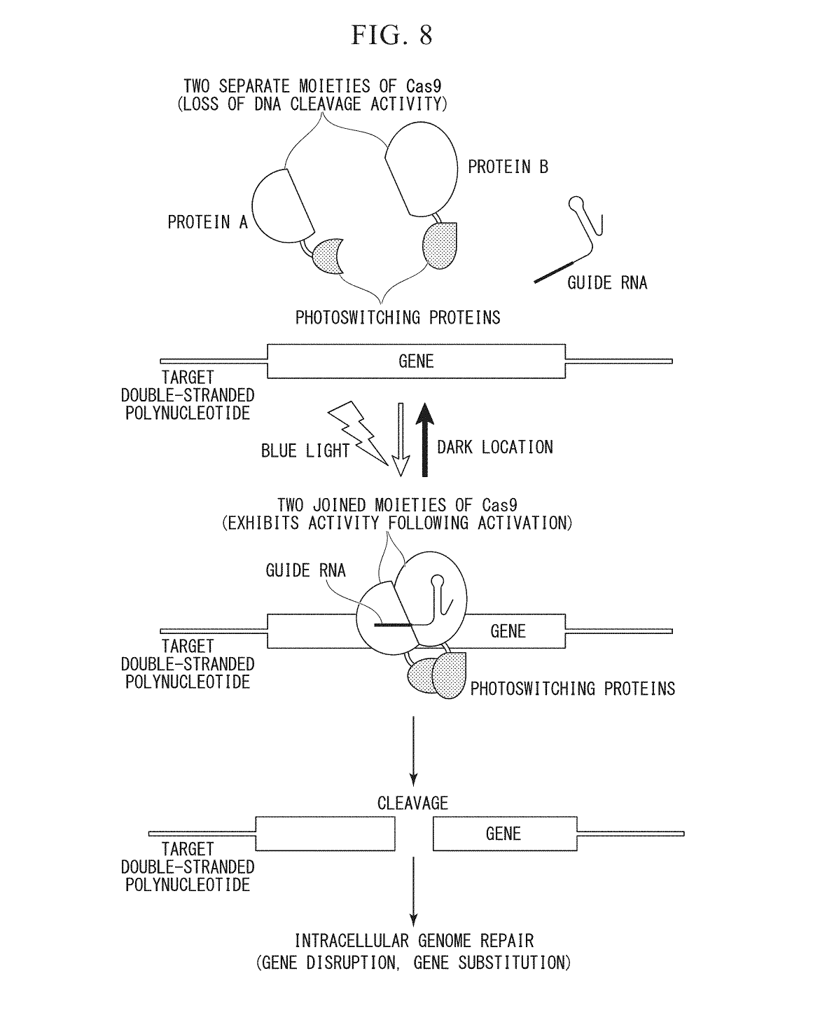

The following provides a detailed explanation of a method for site-specifically modifying a target double-stranded polynucleotide in cells. FIG. 8 is a drawing indicating the steps of a method for site-specifically modifying a target double-stranded polynucleotide in a cell in the present embodiment.

First, the aforementioned protein A, the aforementioned protein B and the aforementioned guide RNA are injected into a cell. The mixture of the protein A, the protein B and the guide RNA is preferably used by suspending in a buffer such as phosphate-buffered saline (PBS).

The injection method can be determined by a person with ordinary skill in the art in accordance with a known method corresponding to the cells used.

Next, the cells are irradiated with blue light having a wavelength of 450 nm to 495 nm. As a result, the photoswitching protein a in the protein A and the photoswitching protein b in the protein B bind, the Cas9 protein is reconstructed, and the RNA-guided DNA endonuclease activity thereof can be restored (switched on).

In addition, when irradiation with blue light is discontinued, binding strength between the photoswitching protein a in the protein A and the photoswitching protein b in the protein B is no longer present. Consequently, the protein A and the protein B are separated and RNA-guided DNA endonuclease activity is no longer demonstrated (switched off).

Accordingly, since the duration of RNA-guided DNA endonuclease activity can be controlled to be extremely short by controlling the duration of irradiation with blue light, the problem of off-target effects (problem in which cleavage of a double-stranded polynucleotide and modification of the base sequence end up occurring at a site other than the target site) can be reduced, thereby enabling the target double-stranded polynucleotide to be cleaved by Cas9 protein only at the desired timing and desired times.

Other detailed conditions can be determined by referring to the method described in “Nature Biotechnology (2015), ‘Photoactivatable CRISPR-Cas9 for optogenetic genome editing’, doi: 10. 1038/nbt.3245”.

Continuing, a target double-stranded polynucleotide subjected to modification corresponding to an objective can be obtained in a region determined by complementary binding between the guide RNA and the target double-stranded polynucleotide.

<Method for Producing Target Gene Knockout Cells>

According to the method of the present embodiment, cells can be easily produced in which the function of a target gene has been disrupted (knocked out).

According to the method of the present embodiment, cells in which the function of a target gene has been disrupted (knocked out) can be easily prepared.

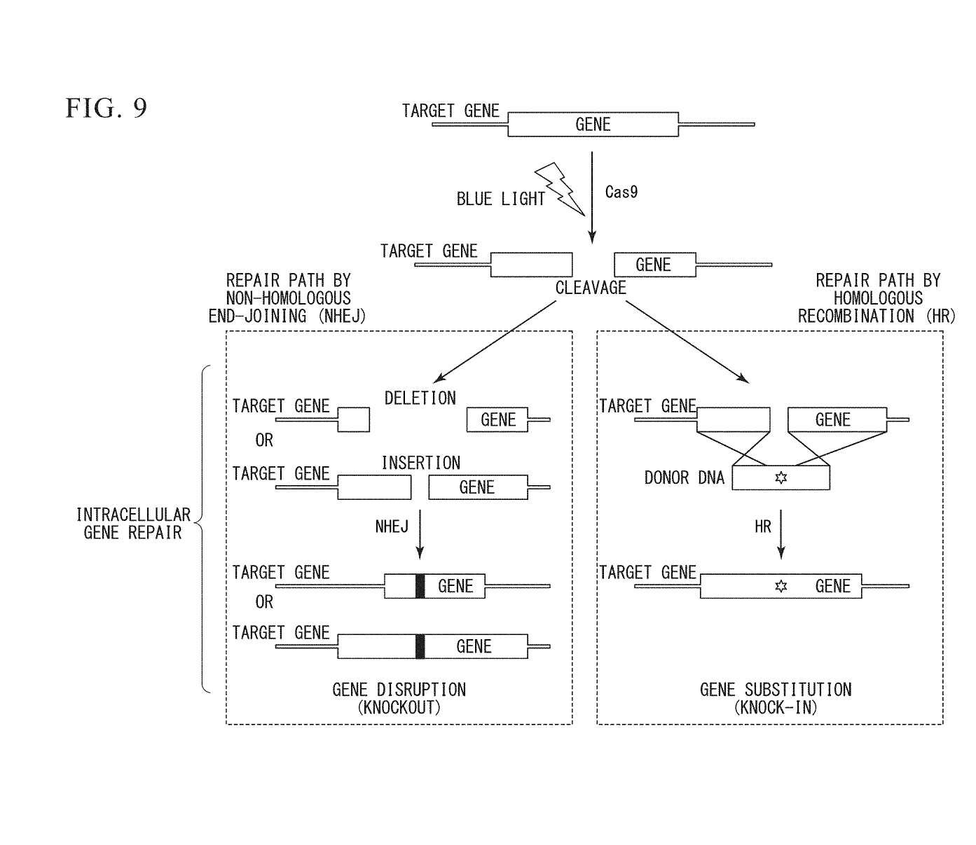

In the present embodiment, the procedure for producing target gene knockout cells is as described in the previous section on <Method for Site-Specifically Modifying Target Double-Stranded Polynucleotide in Cells>. FIG. 9 is a drawing for explaining cleavage of a base sequence in a target gene and the subsequent repair of the target gene in the present embodiment. In the cleaved target gene, base deletion and insertion occurs on the end of the DNA prior to the occurrence of non-homologous end-joining (NHEJ). Accordingly, in a target gene that has been repaired by NHEJ, the function of the gene located at the cleavage site is disrupted (knocked out).

Determination of whether the gene has been knocked out can be confirmed by PCR, sequence determination and the like.

<Method for Producing Target Gene Knock-in Cells>

In one embodiment thereof, the present invention provides a method for producing target gene knock-in cells using the aforementioned method for site-specifically modifying a target double-stranded polynucleotide in cells.

According to the method of the present embodiment, cells can be easily produced in which the function of a target gene is substituted (knocked in).

In the present embodiment, the procedure for producing target gene knockout cells is as described in the previous section on <Method for Site-Specifically Modifying Target Double-Stranded Polynucleotide in Cells>. FIG. 9 is a drawing for explaining cleavage of a base sequence in a target gene and the subsequent repair of the target gene in the present embodiment. As a result of inserting DNA referred to as donor DNA into a cell either simultaneous to, or before or after, introduction of Cas9 protein, having a sequence having a high degree of homology with the cleavage site of the target gene, and guide RNA into the cell, homologous recombination (HR) occurs between the cleavage site of the genome and the donor DNA. In the target gene repaired by HR, the original base sequence of the gene is substituted (knocked in) with the base sequence of the donor DNA. Determination of whether the target gene has been knocked in can be confirmed by PCR, sequence determination and the like.

<Gene Therapy>

In one embodiment thereof, the present invention provides a method and composition for gene therapy by carrying out genome editing. In contrast to previously known methods for targeted gene recombination, the method of the present embodiment can be carried out efficiently and inexpensively and can be applied to any cell or living organism. An arbitrary segment of a double-stranded nucleic acid of a cell or living organism can be modified by the gene therapy method of the present embodiment. The gene therapy method of the present embodiment utilizes both homologous and non-homologous recombination processes present in all cells.

In the present description, the term “genome editing” refers to a novel gene modification technology for carrying out a specific gene disruption or knock-in of a reporter gene by carrying out targeted recombination or targeted mutation using a technology such as the CRISPR/Cas9 system or transcription activator-like effector nucleases (TALEN).

In addition, in one embodiment thereof, the present invention provides a gene therapy method for carrying out targeted DNA insertion or targeted DNA deletion. This gene therapy method includes a step for transforming a cell using a nucleic acid construct containing donor DNA. The scheme relating to DNA insertion or DNA deletion after cleaving a target gene can be determined by a person with ordinary skill in the art in accordance with a known method.

In addition, in one embodiment thereof, the present invention provides a gene therapy method for carrying out gene manipulation at a specific genetic locus using both somatic cells and germ cells.

In addition, in one embodiment thereof, the present invention provides a gene therapy method for disrupting a gene in a somatic cell. Here, the gene expresses a product harmful to cells or living organisms by over-expressing a substance harmful to cells or living organisms. This type of gene is over-expressed in one or more cell types generated in a disease. Disruption of the aforementioned over-expressed gene by the gene therapy method of the present embodiment is able to bring about a more favorable state of health in an individual suffering from a disease attributable to the aforementioned over-expressed gene. Namely, therapeutic effects are manifested as a result of the gene being disrupted in only a very small proportion of cells, thereby leading to a reduction in the expression level thereof.

In addition, in one embodiment thereof, the present invention provides a gene therapy method for disrupting a gene in a germ cell. Cells in which a specific gene has been disrupted can be used to create living organisms that do not have the function of a specific gene. A gene can be completely knocked out in cells in which the aforementioned gene has been disrupted. This functional deficit in a specific cell can have a therapeutic effect.

In addition, in one embodiment thereof, the present invention provides a gene therapy method for inserting a donor DNA encoding a gene product. This gene product has a therapeutic effect in the case of having been constitutively expressed. An example of such a method consists of inserting donor DNA encoding an active promoter and insulin gene into an individual suffering from diabetes in order to induce insertion of the donor DNA in an individual group of pancreas cells. Next, the aforementioned individual group of pancreas cells containing exogenous DNA produces insulin making it possible to treat the diabetes patient.

Moreover, a drug-related gene product can be made to be produced by inserting the aforementioned donor DNA into a plant. A gene of a protein product (such as insulin, lipase or hemoglobin) is inserted into the plant along with a control element (constitutively activated promoter or inducible promoter) to enable a large amount of a pharmaceutical to be produced in the plant. Next, this protein product is isolated from the plant.

Transgenic plants or transgenic animals can be produced by methods using nucleic acid transfer technology (McCreath, K. J. et al. (2000), Nature 405: 1066-1069; Polejaeva, I. A. et al. (2000), Nature 407: 86-90). A tissue type-specific vector or cell type-specific vector can be used to provide gene expression only in selected cells.

In addition, in the case of using the aforementioned method in germ cells, cells can be produced having a designed genetic alteration by inserting donor DNA into a target gene and allowing all of the subsequent cells to undergo cell division.

Examples of application targets of the gene therapy method of the present invention include, but are not limited to, any living organisms, cultured cells, cultured tissue, cultured nuclei (including cells, tissue or nuclei able to be used to regenerate a living organism in cultured cells, cultured tissue or intact cultured nuclei) and gametes (such as ova or sperm in various stages of development).

Examples of the origins of cells targeted for application of the gene therapy method of the present embodiment include, but are not limited to, any living organisms (such as insects, fungi, rodents, cows, sheep, goats, chickens and other agriculturally important animals along with other mammals (such as dogs, cats or humans, although not limited thereto)).

Moreover, the gene therapy method of the present embodiment can be used in plants. There are no particular limitations on those plants targeted for application of the gene therapy method of the present embodiment, and the gene therapy method of the present embodiment can be applied to various arbitrary plant species (such as monocotyledons or dicotyledons).

Although the above has provided a detailed description of embodiments of this invention with reference to the drawings, specific configurations thereof are not limited to these embodiments, but rather include configurations within a range that does not deviate from the gist of this invention.

### EXAMPLES

Although the following provides a more detailed description of the present invention by indicating examples and comparative examples thereof, the present invention is not limited to these examples.

### Example 1

1. Preparation of Wild-Type and Mutant FnCas9

(1) Construct Design

FnCas9 genes in which codons had been optimized by gene synthesis (wild-type FnCas9 base sequence: SEQ ID NO: 9, E1369R/E1449H/R1556Amutant FnCas9 base sequence: SEQ ID NO: 10) were respectively incorporated in pE-SUMO vectors (LifeSensors, Inc.). Moreover, a TEV recognition sequence was added between the SUMO tag and FnCas9 gene. The design of the construct was such that six histidine residues (His tag) were linked followed by the addition of the SUMO tag and TEV protease recognition site to the N-terminal of the Cas9 expressed by the completed construct.

Furthermore, the base sequence artificially synthesized by optimizing the human codon by Feng Zhang Lab was used for the base sequence of the wild-type FnCas9.

(2) Expression in E. coli

The resulting vectors were used to transform Escherichia coli strain rosetta 2 (DE3). Subsequently, the E. coli were cultured in LB medium containing 20 μg/ml of kanamycin and 20 μg/ml of chloramphenicol. After having cultured to OD=0.8, an expression inducing agent in the form of isopropyl-β-D-1-thiogalactopyranoside (IPTG) (concentration: 1 mM) was added followed by culturing for 4 hours at 37° C. Following culturing, the E. coli were recovered by centrifugation (5,000 g, 10 minutes).

(3) Purification of Wild-Type and Mutant FnCas9

The bacterial cells recovered in step (2) were suspended in a Buffer A and subjected to sonication. Supernatant was recovered by centrifugation (25,000 g, 30 minutes) followed by mixing with Ni-NTA Superflow Resin (Qiagen Inc.) equilibrated with a Buffer C and gently inverting for 1 hour. After recovering the effluent fraction, the column was washed with the Buffer C in an amount equal to four times the column volume and a high salt concentration Buffer D in an amount equal to two times the column volume.

Next, after again washing with the Buffer C using an amount equal to twice the column volume, the target protein was eluted with a high imidazole concentration Buffer B in an amount equal to five times the column volume. TEV protease was added to the eluted protein followed by dialyzing overnight at 4° C. using Buffer C to remove the tag. Following dialysis, the protein was again mixed with Ni-NTA Superflow resin equilibrated with Buffer C followed by recovering the effluent fraction in order to separate the His tag and TEV protease from the target protein. Continuing, the column was washed with Buffer C in an amount equal to three times the column volume followed by recovery of the washings.

Next, after diluting so that the NaCl concentration of the crudely purified sample was 150 mM, the sample was charged into MonoS (GE Healthcare Inc.) equilibrated with 92.5% Buffer E (0 M NaCl) and 7.5% Buffer F (2 M NaCl). Next, after washing with a mixture of 92.5% Buffer E (0 M NaCl) and 7.5% Buffer F (2 M NaCl) in an amount equal to five times the column volume, the target protein was eluted by applying a linear gradient in which the concentration of Buffer F increased from 7.5% to 50% (NaCl concentration increased from 150 mM to 1 M). Next, the eluted sample was passed through the HiLoad 16/600 Superdex 200 column (GE Healthcare Inc.) and the target protein was eluted with Buffer G in an amount equal to one column volume.

The compositions of Buffers A to G are shown in Table 1. In Table 1, “2-ME” refers to 2-mercaptoethanol, “DTT” refers to dithiothreitol, and “PMSF” refers to phenylmethylsulfonyl fluoride.

TABLE 1

| Buffer | | | Reducing | |
|---|---|---|---|---|
| Solution | Buffer | Salt | Agent | Other |
| A | 20 mM Tris-HCl | 300 mM | 3 mM 2-ME | 20 mM |
| | (pH 8.0) | NaCl | | imidazole, |
| | | | | 0.1M PMSF |
| B | 20 mM Tris-HCl | 300 mM | 3 mM 2-ME | 300 mM |
| | (pH 8.0) | NaCl | | imidazole |
| C | 20 mM Tris-HCl | 300 mM | 3 mM 2-ME | 20 mM |
| | (pH 8.0) | NaCl | | imidazole |
| D | 20 mM Tris-HCl | 1M | 3 mM 2-ME | 20 mM |
| | (pH 8.0) | NaCl | | imidazole |
| E | 20 mM Tris-HCl | | 3 mM 2-ME | |
| | (pH 8.0) | | | |
| F | 20 mM Tris-HCl | 2M | 3 mM 2-ME | |
| | (pH 8.0) | NaCl | | |
| G | 10 mM Tris-HCl | 150 mM | 1 mM DTT | |
| | (pH 8.0) | NaCl | | |

2. Preparation of Guide RNA

A vector inserted with the target guide RNA sequence (SEQ ID NO: 11) was prepared. A T7 promoter sequence was added upstream from the guide RNA sequence followed by incorporating a linearized pUC119 vector (Takara Corp.). Template DNA for an in vitro transcription reaction was produced using PCR based on the resulting vector. An in vitro transcription reaction was carried out by T7 RNA polymerase for 4 hours at 37° C. using this DNA template. After adding an equal volume of phenol-chloroform to the reaction solution containing the gene product and mixing, the solution was centrifuged at 20° C. (10,000 g, 2 minutes) to recover the supernatant. 1/10th volume of 3 M sodium acetate and 2.5 volumes of 100% ethanol were added to the supernatant followed by centrifuging at 4° C. (10,000 g, 3 minutes) to precipitate the transcription product. The supernatant was discarded followed by adding 70% ethanol, centrifuging at 4° C. (10,000 g, 3 minutes) and again discarding the supernatant. After allowing the precipitate to air-dry, the precipitate was re-suspended in TBE buffer and purified by 7 M urea-denatured PAGE. The band located at the molecular weight of the target RNA was cut out and the RNA was extracted with the Elutrap electroelution system (GE Healthcare Inc.). Subsequently, the eluted RNA was passed through a PD-10 column (GE Healthcare Inc.) and the buffer solution was replaced with Buffer H (10 mM Tris-HCl (pH 8.0), 150 mM NaCl).

3. Plasmid DNA Cleavage Activity Measurement Test

Vectors inserted with the target DNA sequence and PAM sequence were prepared for use in a DNA cleavage activity measurement test. PAM sequences 1 to 7 were each added to the target DNA sequence and incorporated in a linearized pUC119 vector. The target DNA sequence and PAM sequences 1 to 7 are shown in Table 2.

TABLE 2

| | | SEQ |
|---|---|---|
| | Base Sequence | ID NO: |
| Target DNA | 5′-GGAAATTAGGTGCGCTTGGC-3′ | 12 |
| PAM Sequence 1 | 5′-TGA-3′ | — |
| PAM Sequence 2 | 5′-TGT-3′ | — |
| PAM Sequence 3 | 5′-TGG-3′ | — |
| PAM Sequence 4 | 5′-TGC-3′ | — |
| PAM Sequence 5 | 5′-AGG-3′ | — |
| PAM Sequence 6 | 5′-GGG-3′ | — |
| PAM Sequence 7 | 5′-CGG-3′ | — |

E. coli strain Mach1 (Life Technologies Corp.) was transformed using the prepared vectors followed by culturing at 37° C. in LB medium containing 20 μg/ml of ampicillin.

Following culturing, the bacterial cells were recovered by centrifugation (8,000 g, 1 minute) and the plasmid DNA was purified using the QIAprep Spin Miniprep Kit (Qiagen Inc.).

A cleavage experiment was carried out using the purified target plasmid DNA containing the seven types of PAM sequences. The plasmid DNA was linearized into a single strand with restrictase BamHI. When the wild-type or mutant FnCas9 was cleaved from the target DNA sequence in this linearized DNA, approximately 1000 bp and 2000 bp cleavage products were obtained. The reaction solutions were allowed to react for 1 hour at 37° C. The compositions of the reaction solutions are shown in Table 3.

TABLE 3

| | Amount Used | |
|---|---|---|
| Wild-type/mutant FnCas9 | 0.1 | pmol |
| Guide RNA | 1 | pmol |
| 10x K buffer (Takara) | 1 | μl |
| BamHI | 0.5 | μl |
| pUC119 | 150 | ng |

| | |
|---|---|
| MilliQ | Up to 10 μl |

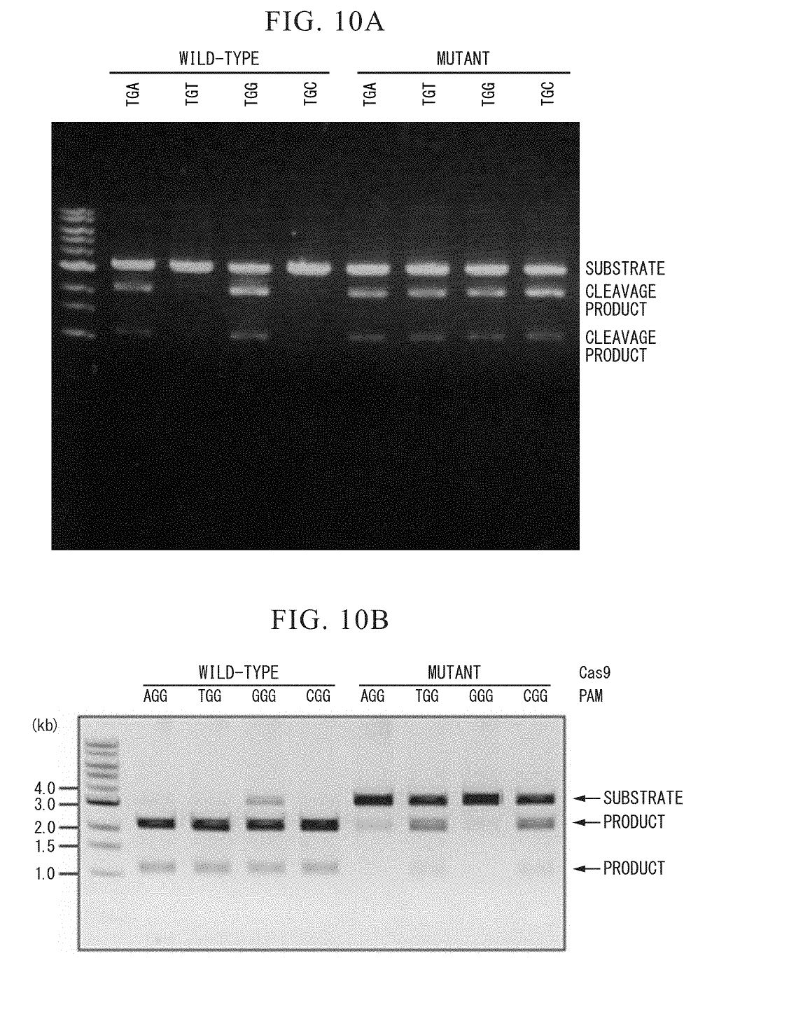

The samples were electrophoresed using agarose gel having a concentration of 1% following the reaction, and bands corresponding to the cleavage products were confirmed. The results are shown in FIGS. 10A and 10B. In FIG. 10B, “Substrate” indicates the substrate while “Product” indicates the cleavage products.

Based on the results shown in FIG. 10A, in contrast to the target plasmid DNA having been cleaved as a result of recognizing only TGA and TGG for the PAM sequence in the case of the wild-type FnCas9, in the case of the mutant FnCas9, the target plasmid DNA was cleaved as a result of recognizing all of the PAM sequences.

In addition, based on the results of FIG. 10B, in contrast to the target plasmid DNA having been cleaved as a result of recognizing all of the PAM sequences in the case of the wild-type FnCas9, in the case of the mutant FnCas9, the target plasmid DNA was cleaved as a result of recognizing only TGG and CGG for the PAM sequence.

Accordingly, in contrast to recognizing “NGR” for the PAM sequence in the case of the wild-type FnCas9, the PAM sequence “YG” was confirmed to be recognized in the case of the mutant FnCas9.

Based on the above results, mutant FnCas9 was determined to be able to recognize a wide range of PAM sequences and site-specifically cleave a target double-stranded polynucleotide for a target sequence both easily and rapidly.

### Example 2

1. Preparation of Mutant FnCas9

Mutant FnCas9 was prepared using the same method as Example 1. SpCas9 (S. pyogenes-derived Cas9) was used as a control and CjCas9 (C. jejuni-derived Cas9) was used as a comparative example.

2. Preparation of Guide RNA

Guide RNA was prepared at lengths of 20 mer, 22 mer and 24 mer each using mouse Tet1 gene (Ex4) for the target gene. Preparation was carried out using the same method as Example 1. The base sequences of the guide RNA are shown in Table 4.

TABLE 4

| | | SEQ |
|---|---|---|
| Guide RNA | Base Sequence | ID NO: |
| Tet1-20 mer | 5′-UGAGCUCCCUGACAGCAGCC-3′ | 13 |
| Tet1-22 mer | 5′-UGAGCUCCCUGACAGCAGCCAC-3′ | 14 |
| Tet1-24 mer | 5′-UGAGCUCCCUGACAGCAGCCACAC-3′ | 15 |

3. Mouse Tet1 Gene (Ex4) Knockout Test

(1) Injection

Solutions were prepared by combining each of the various types of Cas9 and guide RNA of different lengths that were prepared, and diluting with a buffer solution (pH 8.0) composed of 10 mM Tris-HCl and 1 mM EDTA (pH 8.0) followed by injection into fertilized mouse ova.

(2) Confirmation of Development Rates of Fertilized Mouse Ova and Blastocyst Morphology

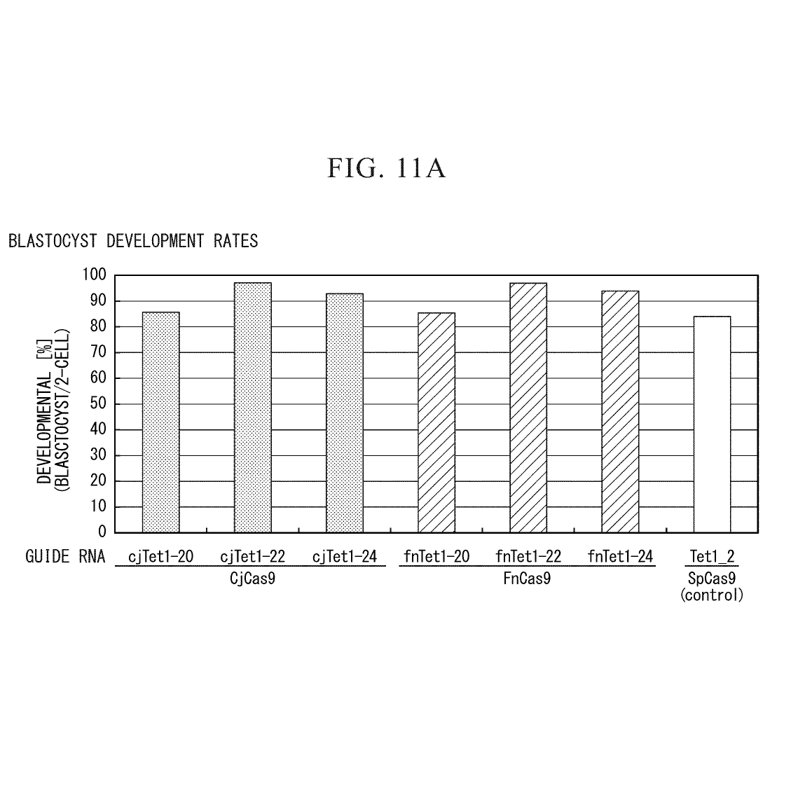

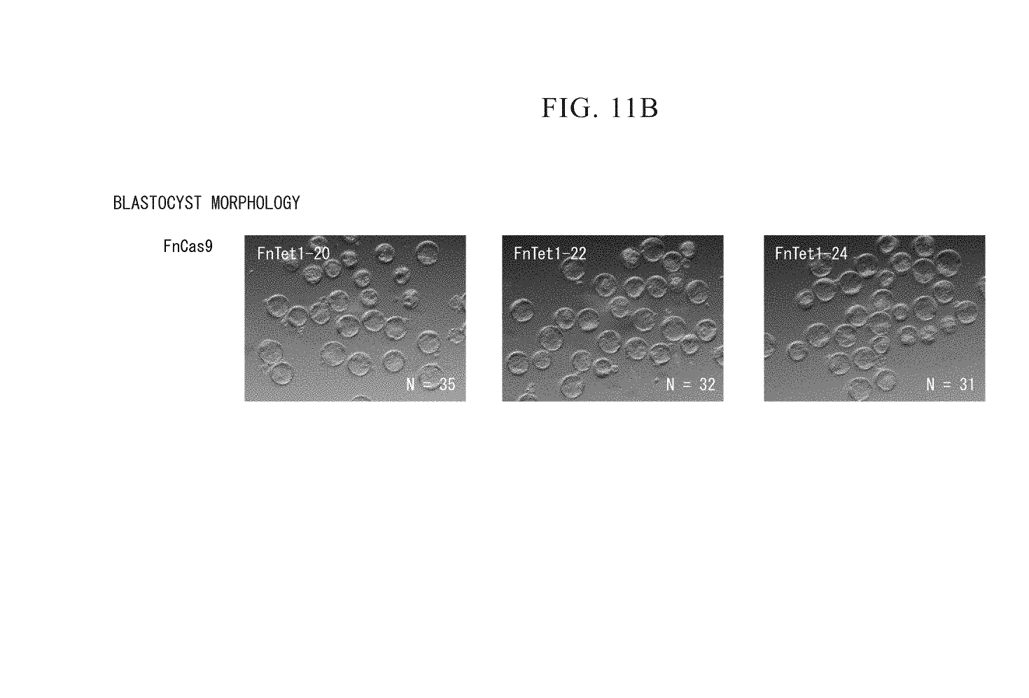

Development rates were confirmed for the resulting blastocysts four days after injection. The results are shown in FIG. 11A. There was no toxicity to ovum development and development rates were favorable. In addition, FIG. 11B depicts images indicating the morphology of blastocysts injected with FnCas9 and guide RNA of different lengths. All of the blastocysts exhibited normal morphology.

(3) Confirmation of Knockout Efficiency of Mouse Tet1 Gene

The blastocysts were recovered four days after injection and knockout efficiency of mouse Tet1 gene was calculated using the method described below. First, genomic DNA was extracted from the cells and the moiety containing the region where knockout was induced by each type of Cas9 was amplified by PCR using primers having the sequences indicated in the following Table 5. Next, cleavage was carried out using restrictase and the success or failure of knockout was evaluated based on the cleavage patterns of the PCR products followed by calculation of knockout efficiency. In the case knockout by each type of Cas9 is successful, the sequence is altered and cleavage of the PCR product by the restrictase does not occur. On the other hand, in the case knockout has not occurred, the PCR product is cleaved by the restrictase. The success or failure of knockout was evaluated based on these cleavage patterns of the PCR products. The results are shown in FIG. 21. The efficiency at which two alleles of mouse Tet1 gene were knocked out in blastocysts injected with the control in the form of SpCas9 and Tet1-20 mer for the guide RNA was assigned a value of 100%.

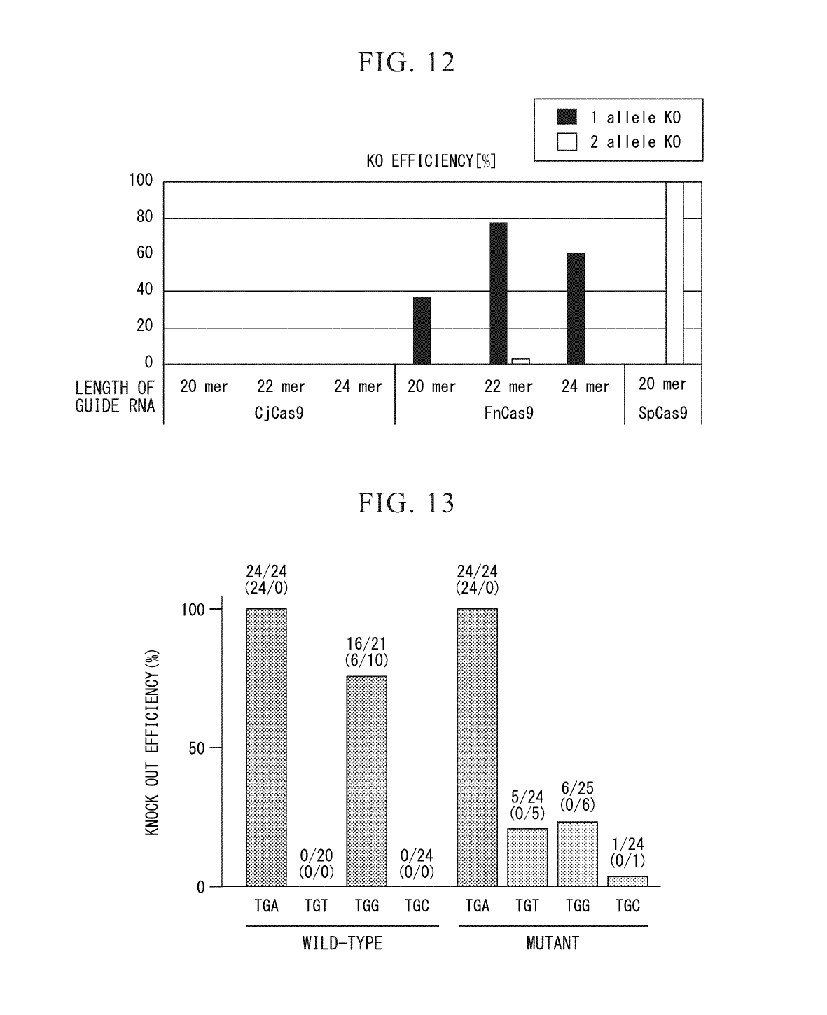

In addition, in FIG. 12, “1 allele KO” indicates knockout efficiency of a single allele of mouse Tet1 gene, while “2 allele knockout” indicates knockout efficiency of two alleles of mouse Tet1 gene.

TABLE 5

| | | SEQ |
|---|---|---|
| Primer | Base Sequence | ID NO: |
| Forward primer | 5′-AGAACAAAGCCCCTGTGCTA-3′ | 16 |
| Reverse primer | 5′-ACCACTCCAAGCCCTTTTCT-3′ | 17 |

On the basis of FIG. 12, injection of mutant FnCas9 and various lengths of guide RNA into fertilized mouse ova made it possible to knock out mouse Tet1 gene. In addition, knockout efficiency was determined to be favorable when the length of the guide RNA was 22 bases.

### Example 3

1. Preparation of Wild-Type and Mutant FnCas9

Wild-type and mutant FnCas9 were prepared using the same method as Example 1.

2. Preparation of Guide RNA

Guide RNA having the base sequences shown in Table 6 were prepared using mouse Tet1 gene (Ex4) for the target gene. The preparation method was the same as that of Example 1.

TABLE 6

| | | SEQ |
|---|---|---|
| Guide RNA | Base Sequence | ID NO: |
| Tet1-TGA | 5′-CCUAGUCUCCAUGAGCUCCCUG-3′ | 18 |
| Tet1-TGT | 5′-CACCUUGGGGCAGGACCAAGUG-3′ | 19 |
| Tet1-TGG | 5′-UGAGCUCCCUGACAGCAGCCAC-3′ | 20 |
| Tet1-TGC | 5′-GAGUUCCUCACCUAGUCUCCAU-3′ | 21 |

3. Mouse Tet1 Gene (Ex4) Knockout Test

(1) Injection

Solutions were prepared by combining the wild-type FnCas9 or mutant FnCas9 and various types of guide RNA following the preparation thereof and diluting with a buffer solution (pH 8.0) composed of 10 mM Tris-HCl and 1 mM EDTA (pH 8.0) followed by injection into fertilized mouse ova.

(2) Confirmation of Development Rates of Fertilized Mouse Ova and Blastocyst Morphology

Development rates were confirmed for the blastocysts four days after injection. There was no toxicity to ovum development and development rates were favorable. In addition, all of the blastocysts exhibited normal morphology.

(3) Confirmation of Knockout Efficiency of Mouse Tet1 Gene

The blastocysts were recovered four days after injection and knockout efficiency of mouse Tet1 gene was calculated using the same method as part (3) of Example 2. The results are shown in FIG. 13. The efficiency at which mouse Tet1 gene was knocked out in blastocysts injected with wild-type FnCas9 and guide RNA was assigned a value of 100%. At this time, knockout efficiency was calculated by counting genes as having been knocked out regardless of whether only one allele or two alleles of mouse Tet1 gene were knocked out.

In addition, in FIG. 13, the numbers shown above the bars of the graph indicate the number of blastocysts in which the gene was knocked out/number of injected fertilized ova, while the numbers shown in parentheses above the bars of the graph indicate the number of blastocysts in which two alleles were knocked out/number of blastocysts in which one allele was knocked out.

Based on the results shown in FIG. 13, in the case of wild-type FnCas9, mouse Tet1 gene was able to be knocked out in the case the PAM sequence was TGA or TGG. On the other hand, in the case of the mutant FnCas9, although there were differences in knockout efficiency, mouse Tet1 gene was able to be knocked out for all PAM sequences.

In addition, in the case of the mutant FnCas9, two alleles of mouse Tet1 gene were knocked out in the case the PAM sequence was TGA, and one allele of mouse Tet1 gene was knocked out in the case of other PAM sequences.

On the basis of these results, the mutant FnCas9 was determined to recognize a wider range of PAM sequences, and use of the mutant FnCas9 was determined to make it possible to easily produce cells in which the function of a target gene had been disrupted (knocked out).

### INDUSTRIAL APPLICABILITY

According to the present invention, a Cas9 protein can be obtained that recognizes a wide range of PAM sequences while retaining binding strength with a target double-stranded polynucleotide and further retaining endonuclease activity. In addition, a simple and rapid site-specific genome editing technology for a target sequence can be provided that uses the aforementioned Cas9 protein.

1. A protein comprising a sequence containing any one of the amino acid sequences of the following (a) to (f) and having RNA-guided DNA endonuclease activity:
(a) amino acid sequence represented by SEQ ID NO: 1,
(b) amino acid sequence in which one or a plurality of amino acids have been deleted, inserted, substituted or added at sites other than amino acid positions 131, 211 and 318 of the amino acid sequence represented by SEQ ID NO: 1,
(c) amino acid sequence having identity of 80% or more at sites other than amino acid positions 131, 211 and 318 of the amino acid sequence represented by SEQ ID NO: 1,
(d) amino acid sequence represented by SEQ ID NO: 2,
(e) amino acid sequence in which one or a plurality of amino acids have been deleted, inserted, substituted or added at sites other than amino acid positions 1369, 1449 and 1556 of the amino acid sequence represented by SEQ ID NO: 2, and
(f) amino acid sequence having identity of 80% or more at sites other than amino acid positions 1369, 1449 and 1556 of the amino acid sequence represented by SEQ ID NO: 2.

2. A gene encoding a protein comprising a sequence containing any one of the base sequences of the following (g) to (j) and having RNA-guided DNA endonuclease activity:
(g) base sequence represented by SEQ ID NO: 3 or 4,
(h) base sequence in which one or a plurality of bases have been deleted, substituted or added in the base sequence represented by SEQ ID NO: 3 or 4,
(i) base sequence having identity of 80% or more with the base sequence represented by SEQ ID NO: 1, and
(j) base sequence capable of hybridizing under stringent conditions with DNA composed of a sequence complementary to DNA composed of the base sequence represented by SEQ ID NO: 3 or 4.

3. A protein-RNA complex provided with the protein according to claim 1 and a polynucleotide composed of a base sequence complementary to a base sequence located 1 to 20 to 24 bases upstream from a proto-spacer adjacent motif (PAM) sequence in a target double-stranded polynucleotide.

4. A method for site-specifically cleaving a target double-stranded polynucleotide, comprising:
a step for mixing and incubating a target double-stranded polynucleotide, a protein and a guide RNA, and
a step for allowing the protein to cleave the target double-stranded polynucleotide at a cleavage site located 3 bases upstream from a PAM sequence to create blunt ends; wherein,
the target double-stranded polynucleotide has a PAM sequence composed of YG (wherein, Y represents a pyrimidine in the form of cytosine or thymine),
the protein is the according to claim 1, and
the guide RNA contains a polynucleotide composed of a base sequence complementary to a base sequence located 1 to 20 to 24 bases upstream from the PAM sequence in the target double-stranded polynucleotide.

5. A method for site-specifically modifying a target double-stranded polynucleotide, comprising:
a step for mixing and incubating a target double-stranded polynucleotide, a protein and a guide RNA,
a step for allowing the protein to cleave the target double-stranded polynucleotide at a cleavage site located 3 bases upstream from a PAM sequence to create blunt ends, and
a step for obtaining a modified target double-stranded polynucleotide in a region determined by complementary binding between the guide RNA and the target double-stranded polynucleotide; wherein,
the target double-stranded polynucleotide has a PAM sequence composed of YG (wherein, Y represents a pyrimidine in the form of cytosine or thymine),
the protein is the protein according to claim 1, and
the guide RNA contains a polynucleotide composed of a base sequence complementary to a base sequence located 1 to 20 to 24 bases upstream from the PAM sequence in the target double-stranded polynucleotide.

6. A method for selectively and site-specifically modifying a target double-stranded polynucleotide in a cell, comprising:
a step for injecting a protein A, a protein B and a guide RNA into a cell,
a step for restoring RNA-guided endonuclease activity by irradiating the cell with blue light to bind the protein A and the protein B,
a step for allowing the conjugate of the protein A and the protein B to cleave the target double-stranded polynucleotide at a cleavage site located 3 bases upstream from a PAM sequence to create blunt ends, and
a step for obtaining a modified target double-stranded polynucleotide in a region determined by complementary binding between the guide RNA and the target double-stranded polynucleotide; wherein,
the target double-stranded polynucleotide has a PAM sequence composed of YG (wherein, Y represents a pyrimidine in the form of cytosine or thymine),
the protein A is a protein having a photoswitching protein a bound to the C-terminal that comprises a protein composed of any of the amino acid sequences of the following (k) to (m) and has RNA-guided DNA endonuclease activity as a result of binding with the protein B:
(k) amino acid sequence represented by SEQ ID NO: 5,
(l) amino acid sequence in which one or a plurality of amino acids have been deleted, inserted, substituted or added in the amino acid sequence represented by SEQ ID NO: 5, and
(m) amino acid sequence having identity of 80% or more with the amino acid sequence represented by SEQ ID NO: 5,
the protein B is a protein having a photoswitching protein b bound to the N-terminal that comprises a protein composed of any of the amino acid sequences of the following (n) to (p) and has RNA-guided DNA endonuclease activity as a result of binding with the protein A:
(n) amino acid sequence represented by SEQ ID NO: 6,
(o) amino acid sequence in which one or a plurality of amino acids have been deleted, inserted, substituted or added at sites other than amino acid positions 526, 606 and 713 of the amino acid sequence represented by SEQ ID NO: 6, and
(p) amino acid sequence having identity of 80% or more at sites other than amino acid positions 526, 606 and 713 of the amino acid sequence represented by SEQ ID NO: 6, and
the guide RNA contains a polynucleotide composed of a base sequence complementary to a base sequence located 1 to 20 to 24 bases upstream from the PAM sequence in the target double-stranded polynucleotide.

7. A method for producing target gene knockout cells using the method according to claim 6.

8. A method for producing target gene knock-in cells using the method according to claim 6.
